(19) United States
(12) Patent Application Publication
(10) Pub. No.: US 2022/0259682 A1
(43) Pub. Date: Aug. 18, 2022

(54) Systems, Methods, And Compositions For The Rapid Early-Detection of Host RNA Biomarkers of Infection And Early Identification of COVID-19 Coronavirus Infection in Humans

(71) Applicant: The Regents of the University of Colorado, a body corporate, Denver, CO (US)

(72) Inventors: Sara L. Sawyer, Boulder, CO (US); Nicholas R. Meyerson, Broomfield, CO (US); Camile L. Paige, Westminster, CO (US); Qing Yang, Longmont, CO (US); Robin Dowell, Broomfield, CO (US)

(21) Appl. No.: 17/686,387

(22) Filed: Mar. 3, 2022

Related U.S. Application Data
(63) Continuation-in-part of application No. PCT/US2020/049290, filed on Sep. 3, 2020.
(60) Provisional application No. 63/006,570, filed on Apr. 7, 2020.
(60) Provisional application No. 62/934,754, filed on Nov. 13, 2019.
(60) Provisional application No. 62/895,387, filed on Sep. 3, 2019.

(51) Int. Cl. C12Q 1/70; C12Q 1/6804

(52) U.S. Cl. CPC C12Q 1/701; C12Q 1/6844; C12Q 1/6804

(57) ABSTRACT

The current inventive technology is directed to systems, methods, and compositions detection of host signatures of pathogenic infection, and in particular a rapid detection assay configured to detect target RNA transcripts that may be biomarkers of infection. In one embodiment, the invention includes systems, methods and compositions for the early detection of pathogens or infection in an asymptomatic subject through a novel lateral flow assay, which in a preferred embodiment may include a rapid self-administered test strip configured to detect one or more RNA transcript biomarkers produced by a subject's innate immune system in response to a pathogen or infection and present in saliva.

This application claims the benefit of and priority to U.S. Provisional Application No. 62/895,387, filed Sep. 3, 2019, and U.S. Provisional Application No. 62/934,754, filed Nov. 13, 2019, and U.S. Provisional Application No. 63/006,570, filed Apr. 7, 2020. The entire specification and figures of the above-referenced applications are hereby incorporated, in their entirety by reference.

### SEQUENCE LISTING

The instant application contains a Sequence Listing which has been submitted electronically in ASCII format and is hereby incorporated by reference in its entirety. Said ASCII copy, created on Aug. 30, 2020, is named “90245.00432-Sequence-Listing.txt” and is 2476 Kbytes in size.

### STATEMENT OF FEDERALLY SPONSORED RESEARCH

This invention was made with government support under grant number HDTRA1-18-1-0032 awarded by Defense Threat Reduction Agency (DTRA). The government has certain rights in the invention.

### TECHNICAL FIELD

The current inventive technology is directed to systems, methods, and compositions detection of host signatures of pathogenic infection, and in particular a rapid detection assay configured to detect target RNA transcripts that may be biomarkers of infection.

### BACKGROUND

Early detection of infection by pathogenic microorganisms is vital for proper treatment and positive clinical outcomes. However, infected individuals may remain asymptomatic for several days post-infection while actively transmitting the pathogen to others. Traditional pathogen detection systems are often not effective at detecting the infection until after the onset of symptoms. Traditional pathogen testing includes serology or antibody-based tests, bacterial/viral/fungal growth cultures, and nucleic acid-based detection such as PCR (polymerase chain reaction). Such traditional tests are often time and labor intensive and are only effective after a patient has begun to show symptoms of the infection. Additionally, traditional diagnostic tests require clinical suspicion for a specific pathogen, expensive laboratory equipment, trained personnel, and have increased upstream and end-user costs.

For example, as highlighted in FIG. 2, in a typical infection course exposure to an unknown pathogen occurs at day zero and then progresses through subsequent clinical stages of infection as indicated by the timeline running vertically along the left side of the figure. As the pathogen replicates within the infected person, standard diagnostic tests are typically designed to work after the onset of symptoms, when people know there is something wrong and seek healthcare and diagnosis. However, at that point the person may have been contagious to others for several days or weeks. The opportunity to implement early quarantine and limit destructive downstream effects of unimpeded pathogen transmission has passed. This time delay to diagnosis can result in poorer patient outcomes and ongoing disease transmission before patients know they are contagious.

As opposed to the specialized, and later developing adaptive immune response, a host's first line of defense against pathogenic microorganisms is the “innate immune” response. The body's innate immunity is a self-amplifying and non-specific physiological response that occurs within hours of infection. As such, the ability to detect the presence of molecules produced by a host's innate immune response may provide the ability to rapidly detect infection at the earliest stages while a patient is still asymptomatic. Such advancement would allow for more effective quarantine protocols, as well as improved treatment and clinical outcomes.

The need for improved methods of detecting pathogens, especially early in the infection cycle, has been magnified by the worldwide coronavirus pandemic. Specifically, in 2019, a novel coronavirus identified as COVID-19, having a high infection and mortality rate, emerged in the Wuhan region of China and later spread throughout the world resulting in sever public health crisis. Coronaviruses, members of the Coronaviridae family and the Coronavirinae subfamily, are found in mammals and birds. A prominent member is severe acute respiratory syndrome coronavirus (SARS-CoV), which killed almost 10% of the affected individuals during an outbreak in China between 2002 and 2003. Another prominent coronaviruses called Middle East Respiratory Syndrome Coronavirus (MERS coronavirus or MERS-CoV) MERS-CoV shares some similarities with the SARS-CoV outbreak. Typical symptoms of a SARS. MERS and COVID-19 coronavirus infection include fever, cough, shortness of breath, pneumonia and gastrointestinal symptoms. Severe illness can lead to respiratory failure that requires mechanical ventilation and support in an intensive care unit. Both coronavirus appears to cause more severe disease in older people, people with weakened immune systems and those with chronic diseases, such as cancer, chronic lung disease and diabetes. At present no vaccine or specific treatment is available for COVID-19. Patients diagnosed with a COVID-19 coronavirus infection merely receive supportive treatment based on the individual's symptoms and clinical condition.

As outlined below, the present inventors have overcome the limitations of traditional pathogen detection systems while leveraging the host's early innate immune response (including but not exclusive to the interferon response) to rapidly detect RNA biomarkers indicative of infection, and particular infection with COVID-19 coronavirus. This rapid point-of-care diagnostic application allows detection of infection at the earlies stages when patients are typically asymptomatic. Such early detection is directly correlated with more targeted and effective therapeutic interventions as well as overall improved clinical outcomes.

### SUMMARY OF THE INVENTION

The inventive technology may include systems, methods and compositions for the early detection of pathogens and/or infection in an asymptomatic subject through a novel lateral flow assay, which in a preferred embodiment may include a rapid test strip configured to detect one or more RNA transcript biomarkers produced by a subject's innate immune system in response to a pathogen or infection and present in saliva.

In another aspect the inventive technology may include systems, methods and compositions for the early detection of pathogens and/or infection in an asymptomatic subject through a novel lateral flow assay, which in a preferred embodiment may include a rapid test strip configured to detect one or more RNA transcript biomarkers encoded by one or more of the nucleotide sequences according to SEQ ID NOs. 1-444, and 657-865 produced by a subject's innate immune system in response to a pathogen or infection, and which may be present in saliva.

Additional aspects of the invention include the use of one or more biomarkers for infection, and preferably pathogen infection in humans according to the nucleotide sequences identified in SEQ ID NOs. 1-444, and 657-865.

In another aspect, the inventive technology may include systems, methods and compositions for the detection of these target RNA transcripts, which may act as biomarkers for early-infection in a subject.

In another aspect, the inventive technology may include systems, methods and compositions for the detection of early-infection in a subject which may include at least: a lateral flow assay test strip device (1) which may preferably include a fibrous or paper-based lateral flow strip (2) configured to allow liquid flow via capillary action; 2) a RT-RPA (reverse transcription recombinase polymerase amplification) reaction which may occur in a pre-prepared reaction cylinder, which may include a collective container configured to receive a fluid sample from a subject and pre-prepared to perform a RT-RPA reaction; and 3) one or more RNA biomarkers transcripts, for example one or more biomarkers encoded by the nucleotide sequences identified as SEQ ID NOs. 1-444, and 657-865, also generally referred to as biomarkers, supplied in a fluid sample, which in a preferred embodiment may include a saliva sample provided by a subject. In a preferred embodiment, an RNA biomarkers transcript may be amplified in a reaction cylinder (3) in an isothermal amplification RT-RPA reaction to form either a hybrid dsDNA probe having single-stranded adapter sequences or a dsDNA product containing 5′ modifications for downstream hybridization.

Additional aspects may include novel conjugated reporter probes (7) that may be coupled with a hybrid dsDNA probe. In certain aspects, a novel conjugated probe may include a GNP, or other single reporter conjugated with a ssDNA sequence or antibody or antibody fragment that may bind to the dsDNA probe. While still, further aspects of the invention may include novel target capture probes that may bind to and form an immobilized “sandwiched” complex aggregate comprising an embedded capture probe coupled with the hybrid dsDNA probe which is further coupled to a conjugated reporter probe (7), and preferably a GNP reporter probe. In this aspect, the localized immobilization may facilitate the generation of a visual signal, for example on a test strip, or even solution.

Additional aspects of the invention include systems, methods, and compositions for the quantification of early host-derived biomarkers of infection that may or may not be combined with quantified data directed to pathogen specific biomarkers, preferably generated by PCR, RT-PCR, or qRT-PCR. In one preferred aspect, RNA may be extracted from a biological sample provided by a potentially exposed or infected subject. The RNA may undergo qRT-PCT reaction to determine the levels of pathogen biomarkers, as well as host-derived biomarkers of infection, and preferably host-derived RNA biomarkers present in the subject's saliva. A plurality of biological samples may be taken from one or more subjects to generate a time-course of infection showing the relative levels of pathogen, and host-derived biomarkers over time. This data may be used to generate biomarker candidates for a lateral flow assay to detect pathogen specific host-derived biomarkers. This lateral flow assay may be administered to a subject in need thereof and provide an indication of infection, as well as the stage of infection by one or more specific pathogens. In one preferred aspect, the specific pathogen may include the SARS-CoV-2, commonly referred to as the COVID-19 coronavirus.

Additional aspects of the invention may include one or more of the preferred embodiments set forth in the claims.

Additional aspects of the invention may be evidenced from the specification, claims and figures provided below.

### BRIEF DESCRIPTION OF DRAWINGS

The novel aspects, features, and advantages of the present disclosure will be better understood from the following detailed descriptions taken in conjunction with the accompanying figures, all of which are given by way of illustration only, and are not limiting the presently disclosed embodiments, in which:

FIGS. 1A-B (A) show a general schematic diagram of a lateral flow assay in one embodiment of the invention thereof; (B) show another general overview of a lateral flow assay test strip in one embodiment of the invention thereof.

FIG. 2 shows a representative example of an infection course.

FIGS. 3A-B (A) shows an exemplary in vivo mouse experiment demonstrating the current state of the art for detection of pathogen infection. In this case, a group of mice may be infected with a pathogen and blood samples will be collected at the indicated days post infection. These samples will be used to carry out high throughput sequencing in order to characterize the presence of biomarkers and may also be used to carry out tests to compare the current invention with current state-of-the art detection methods. Below shows exemplary data showing the invention's ability to detect pathogen infection several days before other methods. All of the illustrated assays will be carried out during prior in vivo experiments. (B) Shows a timeline of a hypothetical viral infection and various tests designed to detect that infection.

FIGS. 4A-C shows an exemplary pathogen detection device in one embodiment thereof and in particular highlights the device's capability for multiplexing. The technology of the invention, and in particular a lateral flow assay test strip or test strip, is adaptable to multiple configurations depending on the aims of the end user. (A) As an initial screening test, the most important parameter is sensitivity to ensure no infected individuals are inadvertently labeled as “not sick” when they are in fact “sick.” A highly sensitive test identifies near 100% of the true positive cases of illness and has a near 0% false negative rate. Sensitivity of RNA transcript biomarker assay is tunable by addition of multiple test lines for different biomarkers, which if detected in combination increases the probability of identifying all true positives. (B) For clinicians assessing already symptomatic patients among diverse medical settings (e.g. emergency departments, primary care offices, assisted care facilities, field hospitals, etc.), it is important to distinguish between the general category of pathogen (i.e. viral vs. bacterial vs fungal) to begin the best early treatment prior to full identification of the causative agent. The inventive assay could inform treatment plans and dramatically reduce the use of antibiotics in cases of non-bacterial infections to help limit the spread of antibiotic resistant bacteria. (C) Early investigation of host signals in response to specific organisms may allow for an assay configuration in which infection by a specific pathogenic organism may be identified. The panel of microbes tested for could be specified by the end users' needs. For example, the military may be most interested in varieties of airborne and weaponizable pathogens while a domestic clinic needs to evaluate patients for seasonal flu, RSV, rhinovirus, and norovirus.

FIG. 5 shows the use of an exemplary pathogen detection device in one embodiment thereof. In this embodiment, the patient provides a saliva sample into a reaction cylinder, which may be represented here as a tube container preloaded with reaction reagents that may allow amplification reaction to proceed at room temperature to increase the biomarker concentration. Following this, the solution containing the amplified biomarkers may be applied to the lateral flow test strip. As fluid flows down the strip, a visible pink signal appears. In the simplest iteration of the strip, one band means a negative result and two bands equal a positive result indicating infection. In a consumer product embodiment, the strip will be contained in housing for ease of results interpretation.

FIGS. 6A-B (A) shows a Venn diagram indicating significant overlap in the identities of RNA transcripts expressed in saliva and PBMCs (peripheral blood mononucleated cells) according to sequencing data of healthy human samples. This overlap implies that transcripts present in the blood are also likely to appear in the saliva. Note, this transcript sequencing data was normalized to an average of 10 million reads coverage and does not describe abundance of these transcripts. (B) Representative PCC (Pattern Correlation Coefficient) Plot showing relative expression levels of RNA transcripts present in both saliva and PBMCs (two samples from the same individual). Every dot in this graph symbolizes a different transcript in the overlapping section of the Venn diagram in A. The average r value=0.64 (>0.5 is considered significant correlation). Overall, there are higher levels of expression of most transcripts in PBMCs vs. saliva, but also a subset of transcripts that are upregulated in saliva relative to PBMCs. Due to this data, the present inventors can pursue saliva as our sample type of choice from which to identify key signals of early infection.

FIG. 7 shows a general approach for identifying biomarkers of infection in one embodiment thereof.

FIG. 8 shows an example of a host RNA biomarker for infection, IFIT2 that was identified using in vitro transcriptomic datasets. Horizontally, the gene structure is shown with dark blue bar indicating the coding region of the gene. Vertically, the height of the peaks represents the relative abundance of the indicated RNA. For each study, the “−” lane indicates non-infected sample, while “+” lane indicates various types of viral infection. The changes in abundance for different studies were highlighted in different colors. Together, the identified RNA biomarker is upregulated across 9 different cell types and 10 different viral infections. The upregulation of this biomarker can be detected in vitro as early as 4 hours post infection which is well prior to any observable symptoms. Additional biomarkers may be identified and selected for use in the invention in a similar procedure as described generally above.

FIG. 9 shows qPCR of biomarker candidates in infected cells. Human lung cells (A549) were mock infected or infected with either influenza virus (left) or vesicular stomatitis virus (VSV, right) for 24 hours. RNA was collected and quantified using qPCR. Results are shown as ‘fold change over mock,’ and a dotted line indicates no change during infection. IFIT2 is an example of an RNA that is global marker of infection, as illustrated in FIG. 8. In this example, NEAT1 would distinguish VSV from influenza, and OAS1 would distinguish influenza from VSV.

FIG. 10 shows a schematic representation of optimization steps used to amplify and detect biomarkers from human saliva. Step 3.1, the RNA from 2 μL human saliva was successfully reverse transcribed into DNA and amplified using a customized RT-RPA kit. The reaction was achieved at constant 37° C. within 20 minutes. Step 3.2, upon successful detection of the potential biomarker for infection, multiple primer sets with different lengths and sequences were designed to optimize the biomarker amplification. The primer set that resulted in the highest amplification efficiency (reflected by the intensity of the band on the gel image) was chosen to be used in actual diagnosis. Step 3.3, the selected primers from previous step is modified to carry adapter sequences to allow downstream hybridization to lateral flow assay test strip and gold nanoparticle reporter probe. After RT-RPA amplification at 37° C. for 20 minutes, the resulting amplicon contains both adapter sequences and the sequences from the target biomarker. The final reaction product can then be directly applied to test strip for visualization.

FIGS. 11A-B demonstrates complementary DNA binding forms nucleic acid “sandwiches” that aggregate for visual readout. The amplified biomarker has a double-stranded DNA (dsDNA) region flanked by specific single-stranded overhanging adapters. The solution with this biomarker is mixed with a gold nanoparticle reporter, which itself is conjugated to a single stranded DNA adapter complementary to adapters of the amplified biomarker and the control capture probe on the nitrocellulose. Due to the mechanism of complementary DNA base pairing, as these overhanging DNA adapter strands interact in solution flowing through the membrane they will bind and form dsDNA structures with the ssDNA conjugated gold nanoparticles and stationary oligo capture probes forming nucleic acid “sandwiches” (FIG. 4A). As more and more of these reporter-amplified biomarker-capture probe sandwich structures form and aggregate, a visible pink signal appears on the nitrocellulose in the target detection zone (B), indicating the presence of that biomarker in the original sample. Here, the leftmost pink dot is representative of the complex illustrated in panel A, and the second pink dot is a control where the gold reporter alone is binding to its complimentary probe. This control verifies that the sample flowed correctly over the strip.

FIG. 12A-C shows colorimetric image of a series of test strips run with 10-fold dilutions of a synthetic RT-RPA product.

FIGS. 13A-D shows a lateral flow assay test strip having an external cover for ease of use in one embodiment thereof.

FIG. 14 shows a general schematic diagram of a lateral flow assay incorporating an antibody-based capture mechanism in one embodiment of the invention thereof.

FIGS. 15A-C shows a general flow diagram of an exemplary laboratory-based test and lateral flow test for detection of biomarkers.

FIG. 16 shows a flow-chart diagram for a designing and validating primers for biomarker candidates. The system being described in U.S. Provisional Application Nos. 62/934,873, and 63/006,561, incorporated herein by references with respect to the disclosure of FIG. 16.

FIG. 17: show host RNA biomarkers are gene transcripts deriving from the earliest immune responses of infected cells. The heatmap was generated from published RNA sequencing datasets and shows the level of expression change (color code at left) of certain RNA species upon infection of cultured human cells with different pathogens (top). In all cases, mock infected (−) and infected (+) cells are compared. Some of the SARS-CoV-2- and Influenza A-specific biomarkers are shown in the orange and green highlighted boxes.

FIGS. 18A-B shows various RNA biomarkers upregulated in response to diverse types of infections and are detectable in human saliva. (A) The heatmap was generated from published RNA sequencing datasets and shows the level of expression change (color code below) of certain RNA species upon infection of cultured human cells with different pathogens (top). (B) In all cases, mock infected (−) and infected (+) cells are compared. Here, we have saliva samples from 3 patients in the infectious disease unit. These represent acute infections with either a fungus (patient 1; Coccidioides), a virus (patient 2; Varicella-zoster virus), and a bacteria (patient 3; E. coli). Quantitative RT-PCR was carried out to measure the fold change of eight of our biomarker RNAs, relative to a healthy saliva control. Note the log scale on the Y-axis, indicating that these biomarkers are found at levels 10-10,000 times higher in the saliva of infected individuals compared to the saliva of healthy individuals. There are also saliva biomarkers that may be able to differentiate one type of infection from others, such as EGR1 which does not respond to fungal infection but is upregulated 100,000-fold in viral infection.

FIG. 19 shows host biomarker upregulation can be detected in a multiplexed RT-qPCR reaction. Human lung cells (A549) were either mock infected or infected with influenza virus and RNA was purified from cell lysates 24 hours after infection. RNA was then subjected to an RT-qPCR reaction using Taqman probes and chemistry. The biomarkers indicated on the X-axis were either measured in singleplex (black bars) or multiplex (orange bars) reactions using the primers and probes listed. Relative mRNA expression (Y-axis) was calculated by first using a host control gene to internally normalize samples, and then compared to the mock infected samples.

FIG. 20 shows some host biomarker upregulation precedes viral RNA detection. A human liver cell line (Huh7) was either mock infected or infected with the SARS-CoV-2 coronavirus. RNA was purified from cell lysates at 0, 2, 4, 8, 12, 24, and 48 hours post infection (X-axis). RNA was then subjected to RT-qPCR using the primers and probes listed. Relative mRNA expression (Y-axis) was calculated by first using a host control gene to internally normalize samples, and then compared to the mock infected samples. A full panel of biomarkers is shown on the left, whereas a subset of biomarkers are shown on the right that highlights biomarkers that are upregulated in the early-stage of infection (blue), late-stage of infection (green), and host control biomarkers that are no upregulated (gray). Detection of the SARS-CoV-2 nucleoprotein gene (N2) is also shown in red.

FIG. 21 show an exemplary lateral flow strip with antibody capturing scheme. Lateral flow strips were striped according to the schematic of FIG. 4 sMimic amplicons were generated in order to test the sensitivity of the lateral flow strip. The ‘excess’ line is capturing excess anti-FITC conjugated gold nanoparticles. The ‘control’ line is capturing mimic amplicons conjugated with FITC and Biotin. The ‘test’ line is capturing mimic amplicons conjugated with FITC and DIG.

FIG. 22 shows Table 3 which includes primers for detecting host biomarkers of infection. A subset of candidate biomarkers was chosen for primer optimization. Listed primer sets were used to carry out RT-qPCR to optimize primer efficiency, Ct values, melting curves, and log fold-change with respect to two host control biomarkers (RACK1 or CALR). Expression in untreated human lung cells (A549) was compared to either interferon treated A549 cells (A549+IFN) or influenza virus infected A549 cells (A549+flu).

FIG. 23 shows s Table which includes primers and probes for multiplexed detection of host biomarkers. A subset of candidate biomarkers from this Table was chosen based on their large fold-changes. Taqman probes were designed for each primer set to be compatible with Taqman fluorescent chemistry in an RT-qPCR reaction. Biomarkers were grouped into triplets based on Ct values in order to be compatible for multiplexing.

FIGS. 24A-B shows a Table which includes primers for amplifying host biomarkers using isothermal RT-RPA. A subset of candidate biomarkers was chosen for optimization of RT-RPA reactions (A). Those primer sets that satisfied conditions presented in FIG. 16 were then modified to contain 5′ modifications (FITC, Biotin, or DIG) for compatibility with the lateral flow assay of the invention (B).

FIGS. 25A-B shows amplified products from RT-RPA reactions can be detected on a lateral flow strip. (A) Lateral flow strips striped with secondary anti-rabbit antibody (gold nanoparticle excess line), streptavidin (control line) or anti-DIG antibody (biomarker line) were used to resolve the indicated RT-RPA reactions. Sample #1 only contains PBS and no RT-RPA reaction products, whereas all the other samples contain RT-RPA reaction (20-minute reaction) products. RT-RPA was carried out using purified RNA from influenza infected human lung cells (A549) as a template. (B) Lateral flow strips as described in panel A were used to confirm that primer sets on their own do does not produce a false positive signal. Indicated primer sets were mixed with PBS at the same concentration of an RT-RPA reaction and run out on the strips.

FIGS. 26A-C shows the kinetics of mRNA accumulation from biomarkers of infection. (A) A549 human lung cells were infected with Influenza A virus at multiplicity of infection (MOI) of 0.1 for 24 hours. Total RNA was harvested from the cells and 100 ng was used as template in a multiplex TaqMan assay. To demonstrate the dynamic range and the signal consistency, the raw Ct values are shown in the top panel, and the resulting fold changes are shown in the bottom panel. The error bar indicates the SEM from 2 biological replicates. (B) Huh7 human liver cells were infected with SARS-CoV-2 at MOI of 0.01 over a time course of 48 hours. Total RNA was harvested 0, 2, 4, 8, 12, 24, and 48 hours post infection. The fold changes of highlighted host mRNAs (top of each graph) were measured by RT-qPCR. Error bars represent the SEM of 3 biological replicates.

FIGS. 27A-C show abundance of mRNA in human saliva can determine whether individuals are infected with SARS-CoV-2 even in the absence of symptoms. (A) Heatmap summarizing mRNA levels from universal response genes in the saliva of SARS-CoV-2-positive individuals. Each infected sample, represented in columns, is compared to the average of 20 uninfected samples to calculate the relative fold change. The viral load in each saliva sample was measured using a separate RT-qPCR assay, and is reported above the heatmap. (B) Scatter plot correlating the fold change of two individual human mRNAs (top) to viral load. Each dot represents a SARS-CoV-2 infected individual. (C) Accuracy of universal response mRNA abundance in saliva to distinguish SARS-CoV-2-infected from uninfected individuals at different levels of viral loads. For each viral load cutoff, RT-qPCR delta Ct values from half of the SARS-CoV-2 positive samples above the cutoff along with half of the non-infected samples were used to train the logistic regression model, while the other half was used for evaluation. The process is bootstrapped for 100 times and the average ROC curve is plotted.

FIGS. 28A-B shows RPA (isothermal amplification) amplicons can be specifically detected on a lateral flow strip. (A) Agarose gel electrophoresis of RPA reactions carried out at 39° C. for 20 minutes (control biomarkers: RACK1 and NCL, infection biomarkers: IFI6, IRF9, and OAS2). Primers targeting the indicated control biomarkers were 5′ modified to contain FITC or biotin, while primers targeting the indicated infection biomarkers were 5′ modified to contain FITC or DIG. NTC=no template control, cDNA=reactions containing cDNA prepared from human cell line RNA. (B) Amplicons from panel A were diluted 1:50 in PBS and then run out on a lateral flow strip. Labeling to the right indicates the position of the excess gold capture strip (anti-rabbit mAb), control biomarker capture strip (streptavidin), and infection biomarker capture strip (anti-DIG mAb).

FIGS. 29A-D shows identification of universal response genes: 69 human genes are consistently upregulated in a broad range of infections performed in tissue culture. (A) Heatmap summarizing the observed abundance of mRNA transcripts from RNA-seq data. Each row represents transcripts corresponding to one of the 69 universal response genes. Each column represents the average expression across all mock (−) or infected (+) replicates combined from all studies on a given pathogen. (B) Number of commonly upregulated genes given any random combination of in vitro infection studies out of the 71 analyzed. From each study, we curated a list of significantly upregulated genes. We then compared these genes between randomly chosen groups of 2-10 studies (x axis). The X axis was truncated at 10 studies, because the analysis has become asymptotic at that point. (C) A characterization of the identified universal response genes via gene ontology enrichment analysis. The adjusted P value indicates the probability of observing the given number of genes in the specific gene ontology term by chance. Functions related specifically to anti-viral responses are the most enriched, and this could be due to an over representation of viral infection studies within the datasets analyzed in panel A, or because innate immunity to viruses is better studied and therefore the genes involved are better annotated. (D) Principal component analysis of gene expression data from the datasets analyzed in panel A. Mock (circles) vs. infected (triangles) samples are separated by the primary principal component (81.6% of data variance) x-axis.

FIGS. 30A-B shows the power of universal response mRNA abundance to identify infected human cells. Receiver operating characteristic (ROC) curves of various linear regression models established using the expression levels of the 69 universal response genes in the 71 in vitro datasets used. The area under curve (AUC) is summarized in each graph. (A) The performance of a model trained on 10% of the samples from the 71 in vitro datasets. The model was them used to classify the other 90% of the samples as mock-infected or infected. The grey lines indicate each replicate of cross validation, while the red curve summarizes the average ROC curve. The mean, minimum and maximum areas under curve (AUC) are indicated. (B) Cross validation analyses between different types of infections. In each case, the classifier was trained on infections of two types (top of graph) and used to predict whether human cells had been infected with the third type of pathogen based solely on the expression level of the 69 universal response genes.

FIG. 31 shows mRNA structure is preserved in human saliva samples. Sashimi plot indicating mRNA structure is preserved during the saliva sample processing and collection, so that the exon regions are preferentially sequenced over the introns. Shown here are saliva samples from 5 individuals, CXCL8 gene is selected as the example.

FIGS. 32A-D show the abundance of mRNA in human saliva can determine whether diverse infections are present in the body. (A) Heatmap showing relative expression of each of the universal response genes in saliva (rows), in transcripts per million (TPM) normalized to row z-score. Each column represents the saliva sample of one individual. (B) Volcano plot of all genes significantly upregulated in all eight infected patients compared to uninfected (DEseq2 Wald test, Fold change ≥2, Adjusted P-value ≤0.01), separated by their fold change in transcript abundance in saliva (infected vs. non-infected) and Benjamini-Hochberg adjusted p-values. The 69 universal-response genes are highlighted in dark red. (C) ROC curve representing the predictive power of the 69 universal response genes to distinguish healthy versus infected individuals. Grey lines indicate individual cross validations, the red line and shaded area indicate the average and variance from all 10 cross validations, respectively. (D) Total RNA from saliva of 3 clinically diagnosed/infected and 3 healthy individuals were used for RT-qPCR with primers recognizing mRNAs from the universal response genes at the bottom. To calculate the fold change within infected saliva samples, their Ct values were normalized to three control genes and then compared to the 3 non-infected saliva samples. Here, the fold change is calculated between the infected individual and each of the non-infected controls, whereas the error bar reflects the stand errors of means (SEM).

FIGS. 33A-C shows the kinetics of transcription from universal response genes. (A) A549 human lung cells were infected with Influenza A virus at multiplicity of infection (MOI) of 0.1 for 24 hours. Total RNA was harvested from the cells and 100 ng was used as template in the multiplex TaqMan assay described. To demonstrate the dynamic range and the signal consistency, the raw Ct values are shown in the top panel, and the resulting fold changes are shown in the bottom panel. The error bar indicates the SEM from 2 biological replicates. (B) Huh7 human liver cells were infected with SARS-CoV-2 at MOI of 0.01 over a time course of 48 hours. Total RNA was harvested 0, 2, 4, 8, 12, 24, and 48 hours post infection. The fold changes of highlighted host mRNAs (top of each graph; red data line) and of the SARS-CoV-2 genome (blue data line) were measured by RT-qPCR. Error bars represent the SEM of 3 biological replicates. (C) To determine the extent of mRNA variation from day to day in human saliva samples, 7 apparently healthy individuals (SS26-SS32) were asked to collect saliva on a daily basis over a period of 11 days. Total RNA was isolated from each sample and used as a template in the multiplex TaqMan assay described. Four of the universal response genes are shown. Error bars represent the SEM of 7 individuals. In all three panels (A-C), Ct value is converted to fold change by normalizing the Ct value to the Ct value of RPP30, and then normalized again to the abundance of mRNA measured in a mock infection or at Day 1 in panel C.

FIG. 34: show host RNA biomarkers are gene transcripts deriving from the earliest immune responses of infected cells. The heatmap was generated from published RNA sequencing datasets, and shows the level of expression change (color code at left) of certain RNA species upon infection of cultured human cells with different pathogens (top). In all cases, mock infected (−) and infected (+) cells are compared. Some of the SARS-CoV-2- and Influenza A-specific biomarkers are shown in the orange and green highlighted boxes.

FIG. 35 show the number of commonly upregulated genes given any random combination of in vitro infection studies. From each individual in vitro infection studies, we curated a list of significantly upregulated genes. We then compared genes that are commonly upregulated genes among randomly chosen groups of 2-10 studies (x axis), where the number of commonly upregulated genes are summarized in each dot, separated by the y-axis. The red box plot summarizes the distribution of the number of intersections among 70 random groupings given the group size (2-10).

FIG. 36 show cross validation of the linear regression classifier based on the universal response genes during viral, fungal, or bacterial in vitro infections. To assess whether the expression changes of the universal response genes are comparable among viral, bacterial, and fungal infections, we established linear regression classifiers using bacterial and fungal infection data and carried out classification on viral infection studies. We then repeat this step to classify fungal and bacterial infections. The ROC curves and the AUC are summarized in the graph.

FIG. 37 Detection of SARS-CoV-2 nucleic acids in human saliva using RT-qPCR. A total of 1,405 university-affiliated individuals were identified to carry SARS-CoV-2 using an RT-qPCR assay. In this assay, the primers targeted the viral N and E genes, and the template was human saliva. The distribution of the viral load within this population is plotted. The curve interpolating the log-normal distribution of viral load in the saliva of these 1,405 individuals was generated to represent the overall mean and variance of the distribution. The relative viral load in saliva (X axis) was quantified via a standard curve created using purified SARS-CoV-2 viruses (not shown).

FIG. 38 show the detection of dengue virus 3 (DENV3) nucleic acids in human saliva. In experimental infections of humans with dengue virus 3 (DENV3), blood and saliva samples were collected from enrollees at days 0, 1, 2, 3, 4, 6, 8, 10 post-infection. The relative viral load in both biospecimens was quantified using RT-qPCR with primers directed at the dengue genome/transcripts and the template being RNA purified from either blood or saliva. The Ct values resulting from RT-qPCR were converted into genomic copies/mL using a standard curve (not shown). The viral genome was detected 4 days or 6 days post initial exposure in blood and saliva, respectively.

FIG. 39 shows detection of the nucleic acids of other respiratory viruses in human saliva. Saliva from anonymous donors was collected on our university campus. The total RNA was harvested from saliva and was subjected to both human and bacterial ribosomal RNA depletion. The processed saliva RNA was sequenced at 30 million read depth on Illumina NovaSeq 6000 platform with 150-bp pair-end read configuration. The sequencing reads were first mapped to human GRCh38.p13 reference genome, and the unmapped reads were subject to metagenomic analysis using the Genomic Origin Through Taxonomic CHAllenge (GOTTCHA v1.0c) software package to identify the microorganism composition using both viral and bacterial non-redundant reference databases. For two of the saliva samples, the sequencing reads that mapped to viral reference database were summarized in the pie charts above, with their relative abundance indicated in percentages. The identified human pathogens (Human respiratory syncytial virus (RSV), and human coronavirus NL-63) are highlighted in red. This proves that the nucleic acids of both of these pathogens can be detected in human saliva.

### DETAILED DESCRIPTION OF INVENTION

The inventive technology may include systems, methods and compositions for the early detection of pathogens and/or infection in an asymptomatic subject through a novel lateral flow assay, which in a preferred embodiment may include a rapid self-administered test strip configured to detect one or more host RNA transcript biomarkers (coding or non-coding) produced by a subject's innate immune system in response to a pathogen or infection and present in saliva.

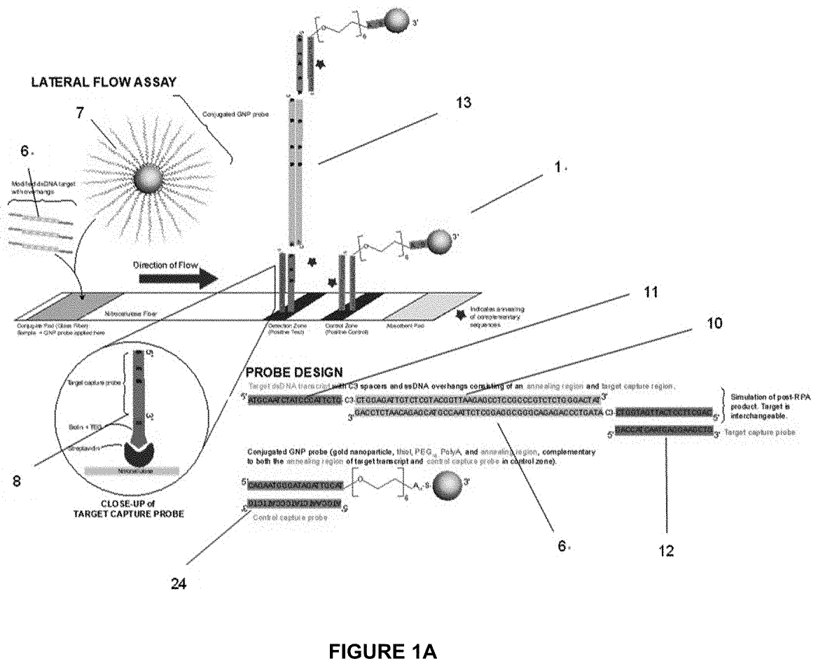

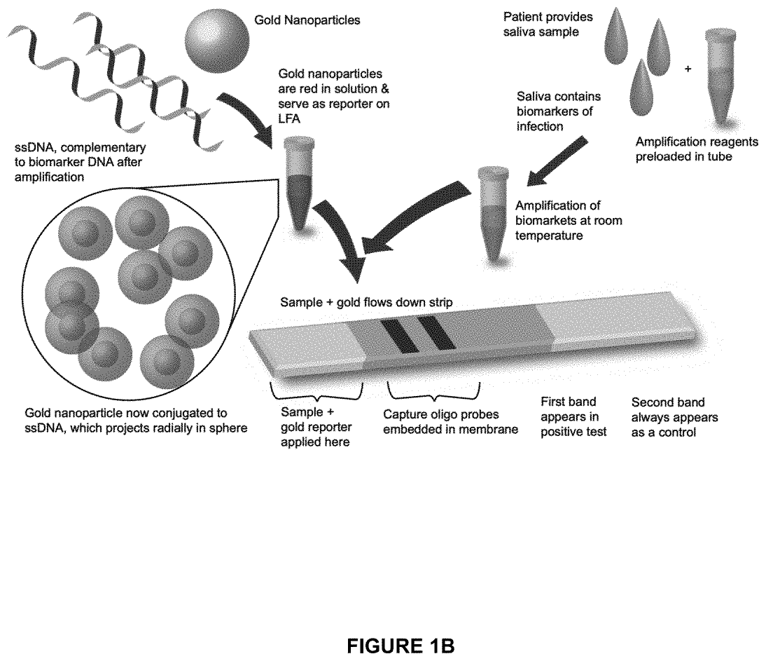

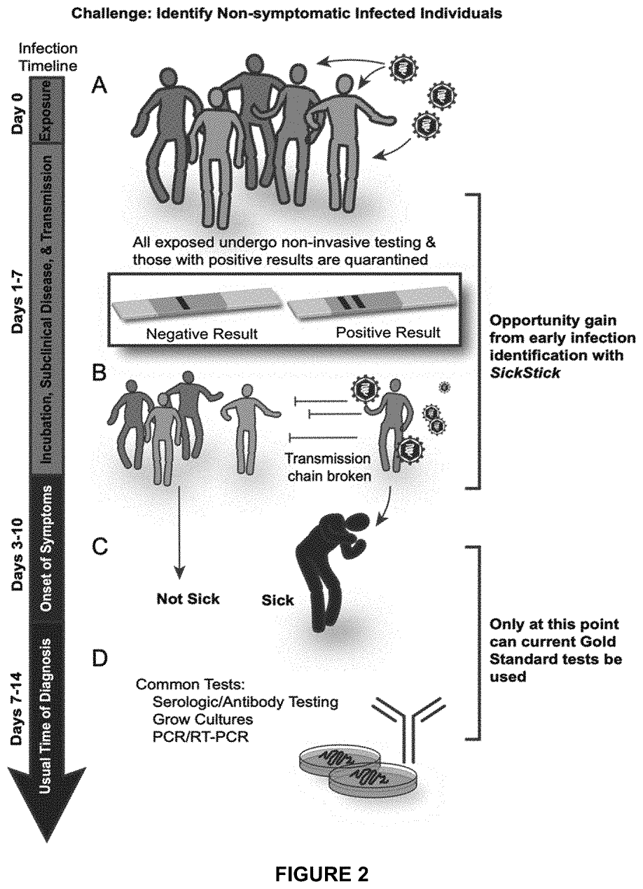

As generally shown in FIG. 1B, one embodiment the inventive technology may include systems, methods and compositions for the detection of early-infection in a subject which may include at least: a lateral flow assay test strip device (1) (also refer to as a test strip, or lateral flow strip), which may preferably include a fibrous or paper-based lateral flow strip (2) configured to allow liquid flow via capillary action; 2) a RT-RPA (reverse transcription recombinase polymerase amplification) reaction which may occur in a pre-prepared reaction cylinder (3), which may include a collective container configured to receive a fluid sample from a subject and pre-prepared to perform a RT-RPA reaction; and 3) one or more RNA biomarkers transcripts, also generally referred to as biomarkers, supplied in a fluid sample, which in a preferred embodiment may include a saliva sample provided by a subject.

Specific target RNA transcripts or biomarkers (9) produced by a patient's immune response (generally innate immune response or any other cellular pathway upregulated upon infection) and found in saliva may be indicative of early infection. As a result, in one embodiment of the inventive technology may include systems, methods and compositions for the detection of these target RNA transcripts, which may act as biomarkers for early-infection in a subject. However, as noted above, target RNA transcript biomarkers present in a typical fluid sample provided by, in this embodiment a human subject, are generally present at low concentrations and require amplification to be detected. To overcome this physical limitation, as further shown in FIG. 1B, in one embodiment of the invention, a subject may deposit a fluid sample, which in this case may comprise a saliva sample, into a reaction cylinder (3) where it may undergo an amplification step. Specifically, a reaction cylinder (3) may receive a fluid sample where it may undergo a RT-RPA reaction to amplify the RNA biomarker transcripts present in a fluid sample. In this preferred embodiment, a reaction cylinder (3) may be pre-loaded with a quantity of pre-prepared proteins, enzymes, salts, and other reagents that may allow for a RT-RPA reaction to proceed within the reaction cylinder. As shown in FIG. 1A, the reaction cylinder (3) may be pre-loaded with primers directed to target RNA biomarker transcripts that may further include C3 spacer elements. In another preferred embodiment, a reaction cylinder (3) may further be pre-loaded with one or more conjugated reporter probes (7), such as a conjugated gold nanoparticle (GNP) reporter probe.

In other embodiments, conjugated reporter probes (7), such as a conjugated gold nanoparticle (GNP) reporter probe may be pre-embedded, dried, lyophilized, or otherwise attached to the conjugate pad instead of being pre-loaded into the reaction cylinder. This specific embodiment may allow for the generation of a lateral flow assay test strip having multiple pre-embedded conjugate pads with different conjugated reporter probes (7).

Again, as shown in FIG. 1B, a fluid sample may be introduced into a reaction cylinder (3) manually by a subject, or through another automated, or semi-automated process, such that one or more RNA biomarker transcripts present in a fluid sample interact with the RT-RPA components, including the modified primers pre-loaded into the reaction cylinder (3) to facilitate a RT-RPA amplifying reaction. Importantly, in this preferred embodiment, the reaction cylinder (3) may be configured to generate the RT-RPA reaction isothermally.

In one embodiment, a reaction cylinder (3) may contain the necessary pre-prepared proteins, enzymes, salts, and other reagents necessary for a RT-RPA reaction to proceed isothermally at approximately room temperature (˜25° C.) or body temperature (˜37° C.) by holding in one's hand, eliminating the need for the laboratory equipment generally required to amplify nucleic acids. In one preferred embodiment, the RT-RPA reaction may proceed in the reaction cylinder (3) for a period of approximately 30 minutes or less.

As highlighted in FIG. 1A, the result of this isothermal RT-RPA reaction may include an engineered probe having a hybrid double stranded DNA (dsDNA) probe of a target biomarker sequence (GREEN (10)) coupled, in this case through a C-3 spacer, with overhanging single-stranded DNA (ssDNA) regions at its 3′ and 5′ ends. A first overhanging ssDNA region, in FIG. 1a at the 5′ end of the dsDNA probe, may include an annealing region (ORANGE (11)), while a second overhanging ssDNA region, shown here at the 5′ end of the dsDNA probe may include a target capture region (BLUE(12)).

Once the RT-RPA reaction is completed, the contents of the reaction cylinder (3) may be introduced to one or more conjugated reporter probes (7), which in a preferred embodiment may act as visual reporters by producing an observable indication of, for example the presence of a target RNA biomarker transcript in a sample. As shown above, a conjugated reporter probe may include a conjugated gold nanoparticle (GNP) (4) conjugated to single stranded DNA (ssDNA) molecule (5) complementary to both the annealing regions of the hybrid double stranded DNA molecules and a control capture probe (24) as discussed below. Naturally, the use of a GNP is exemplary only, as a variety of metalloid nanoparticle reporters of various geometries and sizes may be incorporated into the inventive technology. Additional embodiments may also include one or more non-metalloid reporter probes, such as fluorescence, enzymatic, or antibody reporters.

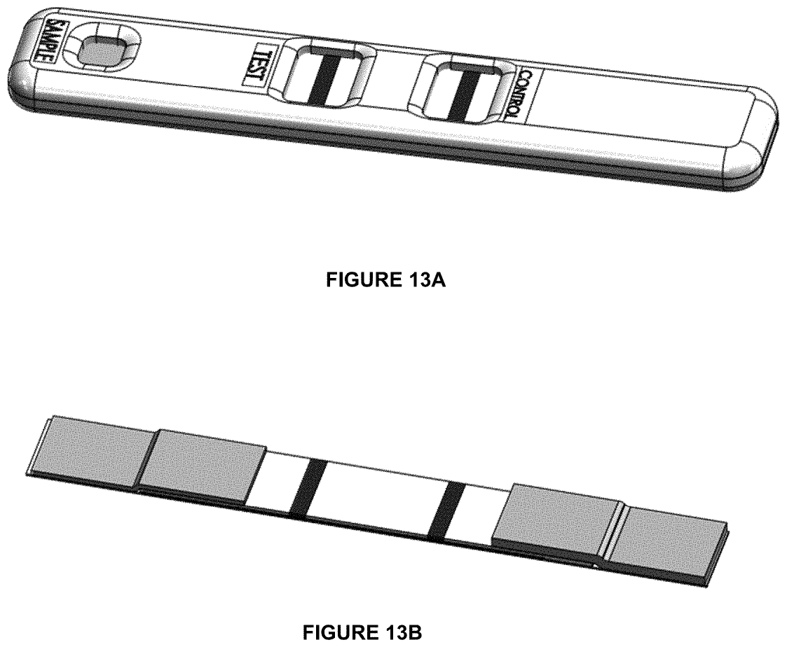

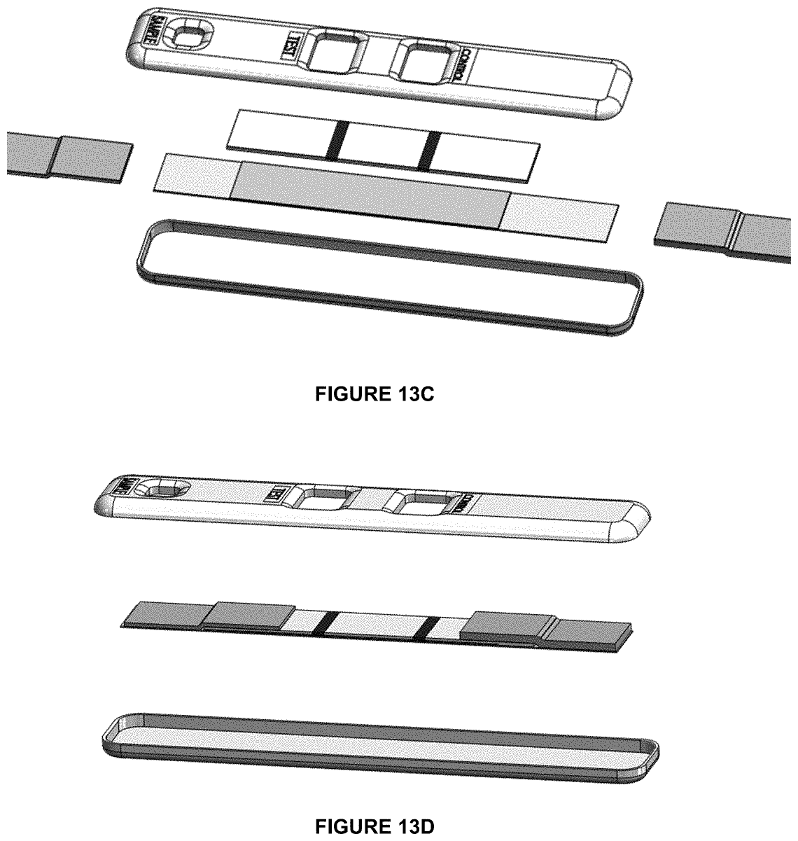

Again, referring to FIG. 1A, in the preferred embodiment highlighted above, this annealing region may be coupled with a GNP through a thiol, PEG₁₈ and PolyA construct. Notably, in this configuration, when a conjugated GNP reporter probes are concentrated in solution or in a small surface area, such as one or more discrete bands on the lateral flow test strip shown in FIG. 13, they may provide a visual signal, which in this embodiment may include a colored band, shown as a red band in FIGS. 1B and 13.

As further shown in FIG. 1B, the hybrid dsDNA probe (6) containing the target dsDNA transcript sequence with an annealing region and target capture region generated in the amplifying reaction in reaction cylinder (3) may be combined with a DNA-conjugated GNP reporter probe. In this embodiment, in the presence of an optimal running buffer, the complementary regions of the hybrid DNA molecule and DNA-conjugated GNP reporter probe may anneal forming an aggregated complex (13). As should be understood from the disclosure, such aggregate complexes may only form if the expected target sequence, in this case a biomarker indicative of early-infection, is both present in the sample and amplified via the RT-RPA reaction localized in the reaction cylinder.

Referring now to FIGS. 1A-B, in a preferred embodiment, the combined solution containing the aggregate complexes formed by the hybrid dsDNA probe (6) coupled with the DNA-conjugated GNP reporter probe may be introduced to the lateral flow strip. In a preferred embodiment, this combined solution may be introduced into a conjugate pad (14) region made preferably of glass fiber. The combined solution may flow via capillary action through a membrane, such as a nitrocellulose fiber membrane, towards an absorbent pad (16) region on the lateral flow strip (2) that may include a detection zone (17) having one or more capture probes embedded to the surface of the lateral flow strip, and preferably the surface of a nitrocellulose membrane (15) of a test strip. The position and orientation of the capture probes embedded in nitrocellulose membrane (15) of a test strip may be adjusted to optimize signal generation or sample-probe interactions. Notably, the absorbent pad (16) region may be positioned at the distal end of the lateral flow strip (2) to facilitate sample flow via capillary action through the detection zone.

As highlighted in FIG. 1A, a capture probe may include an immobilized streptavidin base tetramer (21) embedded in the nitrocellulose surface of a lateral flow strip. This immobilized streptavidin base may be coupled with a biotin-TEG linker (22) that may further be coupled with a ssDNA target capture probe (8) sequence that may be complementary to a target capture region on a hybrid dsDNA probe.

Again, in the preferred embodiment shown in FIG. 1A, the target capture region of a hybrid dsDNA probe (6) may anneal to a complementary capture probe ssDNA sequence (5) forming an immobilized “sandwiched” complex aggregate comprising an embedded capture probe coupled with the hybrid dsDNA probe (6) which is further coupled to the DNA-conjugated GNP reporter probe. As can be seen in FIGS. 1A-1B, where a biomarker of interest is present (i.e. a biomarker indicative of pathogen infection in a subject), the “sandwich” complex may be immobilized at a discrete position along the lateral flow strip. As noted above, the GNP reporter probes of the invention produce a red color signal in solution or when immobilized on the lateral flow strip. As such, when a certain concentration of complex aggregates is captured in close proximity to one another a visible signal within the detection zone (17) may be generated, which in this exemplary embodiment is shown as a red-pink band on the lateral flow strip. This visible signal within the detection zone (17) may indicate a positive result indicating the presence of a target pathogen, or an early-indication of infection in a subject. Notably, this process as generally described above may take less than 10 minutes and, in some instances, less than 3 minutes to run to completion and provide a discernable signal.

As further shown in FIG. 1A, any unbound GNP reporter probes not immobilized within the detection zone (17) may continue to flow through the lateral flow strip (2) towards a distal absorbent pad (16) and anneal to a control capture probe (24) immobilized to a control region on the surface of the lateral flow strip. In this manner, the unbound GNP reporter probes immobilized in the control region will also produce a visible signal providing a positive control for the system.

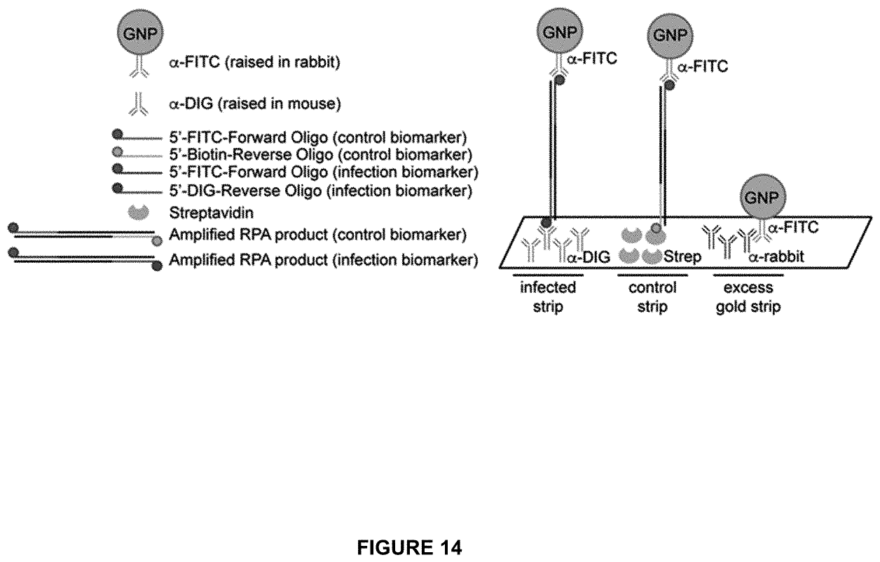

In an alternative embodiment, the invention may include a lateral flow assay strip having an antibody-based capture mechanism. Similar to the lateral flow assay described in FIG. 1A, the result of this isothermal RT-RPA reaction may include an amplified RPA product that may act as a control biomarker, and another amplified RPA product that may act as an infection biomarker. Once the RT-RPA reaction is completed, the contents of the reaction cylinder (3) may be introduced to one or more conjugated antibody reporter probes, which in a preferred embodiment may act as visual reporters by producing an observable indication of, for example the presence of a target RNA biomarker transcript in a sample. More specifically, as shown in FIG. 14, the isothermal RT-RPA reaction may generate at least two amplified RPA products, or amplicons, namely a control biomarker and infection biomarker respectively having modified 5′ ssDNA overhang regions forming a probe capture region and a target capture region respectively. In this embodiment, a control biomarker may include a dsDNA transcript region coupled with a 5′ FITC forward ssDNA oligo (GREEN) and 5′ biotin reverse ssDNA oligo (ORANGE). The infection biomarker of this embodiment may include a dsDNA transcript region coupled with a 5′ FITC forward ssDNA oligo (GREEN and PINK) and a 5′ DIG ssDNA reverse oligo (BLUE).

As further shown in FIG. 14, GNP may be conjugated with an anti-FITC (fluorescein isothiocyanate) antibody, and preferably an anti-FITC antibody (19) produced in a rabbit. As also shown in FIG. 14, streptavidin may also be stripped onto the membrane (15) as generally described above to capture control biomarker amplicons present in the amplified RPA product. In this embodiment, an anti-DIG (Digoxigenin) antibody (20), and preferably an anti-DIG antibody raised in mouse, may also be stripped onto the lateral flow membrane (15) to capture infection biomarker amplicons present in the amplified RPA product.

As further shown in FIG. 14, the hybrid dsDNA control and infection amplicon probes generated in the amplifying reaction may be combined with an anti-FITC antibody-conjugated GNP reporter probe. In this embodiment, the anti-FITC antibody may bind to the 5′ FITC-forward oligo of the control and infection biomarker forming an aggregated complex (13). In this embodiment, the aggregated complexes (13) may further be introduced to the lateral flow strip (2) of the invention. In a preferred embodiment, this combined solution may be introduced into a conjugate pad (14) region made preferably of glass fiber. The combined solution may flow via capillary action through a membrane, such as a nitrocellulose fiber membrane, towards an absorbent pad (16) region on the lateral flow strip (2) that may include a detection zone (17) having one or more capture probes embedded to the surface of the lateral flow strip, and preferably the surface of a nitrocellulose membrane (15) of a test strip. The position and orientation of the capture probes embedded in nitrocellulose membrane (15) of a test strip may be adjusted to optimize signal generation or sample-probe interactions. Notably, the absorbent pad region may be positioned at the distal end of the lateral flow strip (2) to facilitate sample flow via capillary action through the detection zone.

As noted above, a capture probe may include an immobilized streptavidin base tetramer (21) embedded in the nitrocellulose surface of a lateral flow strip. This immobilized streptavidin base may be coupled with a biotin-TEG linker (22) that may further be coupled with a ssDNA target capture probe sequence that may be complementary to a target capture region on a hybrid dsDNA probe, and preferably the 5′ biotin-reverse oligo. Further, a capture probe may include an immobilized anti-DIG antibody that may be configured to bind to the 5′ DIG-reverse oligo. In this configuration, control and infection biomarker amplicons may be bound to their respective locations by their respective capture probes. As noted above, the GNP reporter probes of the invention produce a red color signal in solution or when immobilized on the lateral flow strip. As such, when a certain concentration of complex aggregates are captured in close proximity to one another a visible signal within the detection zone (17) may be generated. This visible signal within the detection zone (17) may indicate a positive result indicating the presence of a target pathogen, or an early-indication of infection in a subject. Notably, this process as generally described above may take less than 10 minutes and, in some instances, less than 3 minutes to run to completion and provide a discernable signal.

As further shown in FIG. 1A, any unbound GNP reporter probes not immobilized within the detection zone may continue to flow through the lateral flow strip (2) towards a distal absorbent pad and anneal to an anti-rabbit control capture probe (23) immobilized to a control region on the surface of the lateral flow strip, being configured to capture unbound antibody-conjugated GNP reporter probe. In this manner, the unbound GNP reporter probes immobilized in the control region may also produce a visible signal providing a positive control for the system.

Naturally, the system may be adapted for a variety of practical applications. For example, the system may be modified to detect a plurality of biomarkers RNA transcripts corresponding with a plurality of distinct capture probes at a plurality of detection zones on a lateral flow strip. Moreover, it should be noted that such probes and their design are exemplary only, as a variety of different probe configurations, as well as probe-generated signals may be interchangeable within the system as generally described herein.

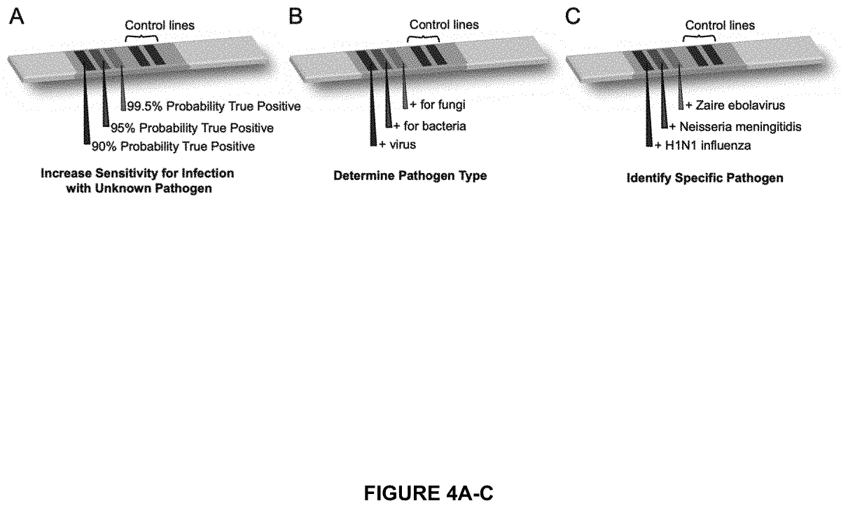

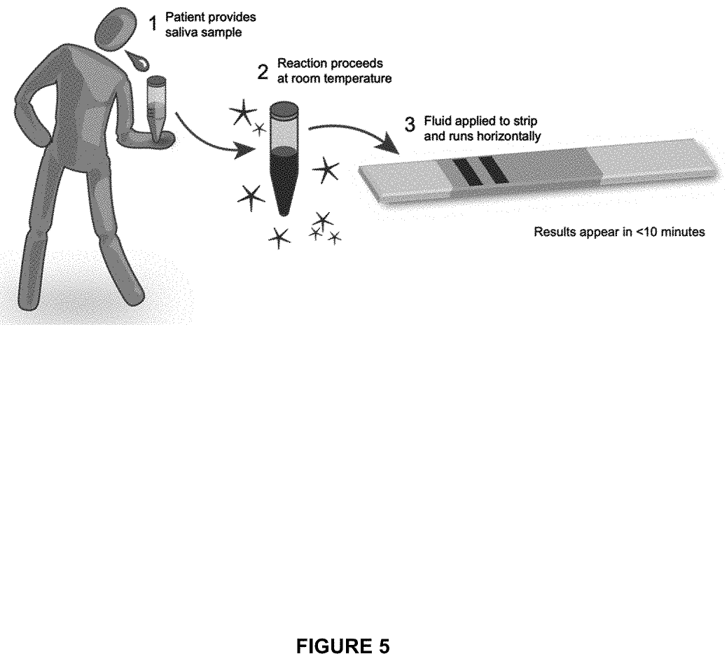

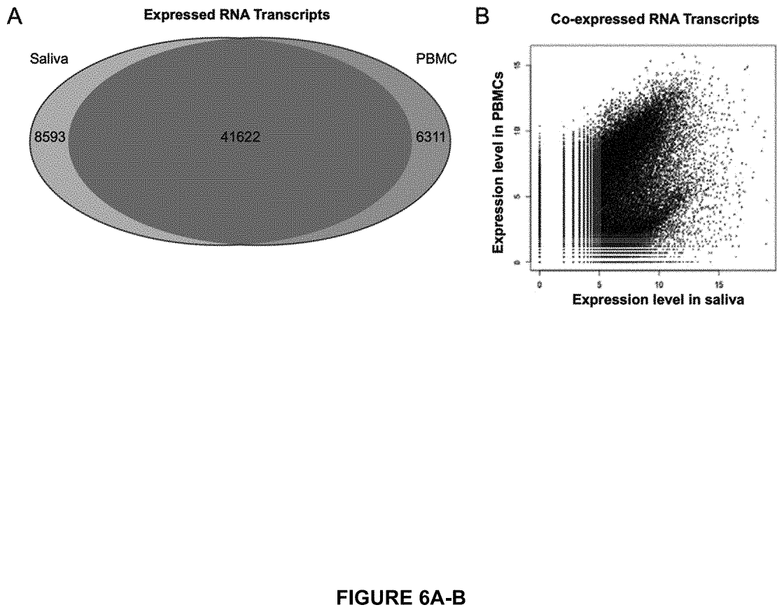

For example, as shown in FIG. 4, in one embodiment, the above described lateral flow detection system may be used to detect, with varying degrees of sensitivity, infection of a subject by a known or unknown pathogen. In other embodiments, the above described lateral flow detection system may be used to determine pathogen type, such as bacteria, virus or fungal. In additional embodiments, the above described lateral flow detection system may be used to determine specific pathogens or their serotypes.

In one embodiment the inventive technology may include novel systems, methods, and composition for the detection of pathogen specific infection in a subject in need thereof. In one preferred embodiment, the inventive technology may provide for the detection of infection of a specific pathogen in a human subject. In this preferred embodiment, a biological sample, which may preferably include a saliva sample, may be provided by a subject which may contain one or more biomarkers for infection with a specific pathogen. In this embodiment, a saliva sample, may be further processed, for example by an on-site, or off-site clinical laboratory wherein RNA molecules present in the saliva sample are extracted for further testing. The extracted RNA is then undergoing a qRT-PCR process where the biomarkers of the pathogen. In the embodiment, one or more of the primer sequencers known to be directed to a components of a target pathogen may be used to identify specific biomarkers produced by the target pathogen. In this embodiment, the subject may provide a plurality of biological samples for RNA extraction and qRT-PCT processing so as to generate a time-course of pathogen biomarkers. These plurality of samples may provide a quantified baseline progression of target pathogen biomarkers from an initial point of exposure to the pathogen in a subject. As can be appreciated from the foregoing, such processes may be implemented for multiple target pathogens, and may further be conducted in series using multiple subjects to generate a library of time-course biomarkers of target pathogens.

As noted above the inventive technology may allow the detection of host-derived biomarkers that may be present in a subject's biological sample before the virus can be detected and well before any symptoms of infection may occur. In one preferred embodiment, RNA may be extracted from the biological sample, which in this case is a saliva sample containing host derived biomarkers of infection and further subject to qRT-PCR. In this embodiment, the subject may provide a plurality of biological samples for RNA extraction and qRT-PCT processing so as to generate a time-course of host-derived biomarkers. Again, multiple samples may provide a quantified baseline progression of host-derived biomarkers, such as RNA biomarkers generated by the hosts innate-immune response in response to the target pathogen from an initial point of exposure to the pathogen and through the incubation period. Again, as can be appreciated from the foregoing, such processes may be implemented for multiple target pathogens, and may further be conducted in series using multiple subjects to generate a library of time-course host-derived biomarkers, and preferably host-derived RNA biomarkers produced in response to a target pathogen. By combining RNA markers from both the host innate-immune response occurring during the incubation period, and from the target pathogen itself, the invention may expand the detection window for infection by various pathogens.

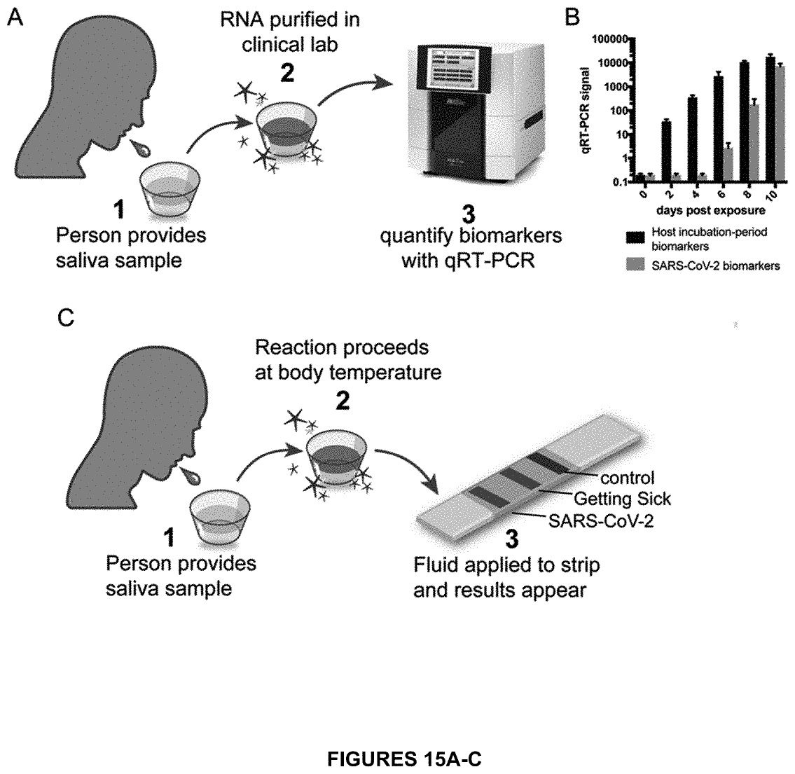

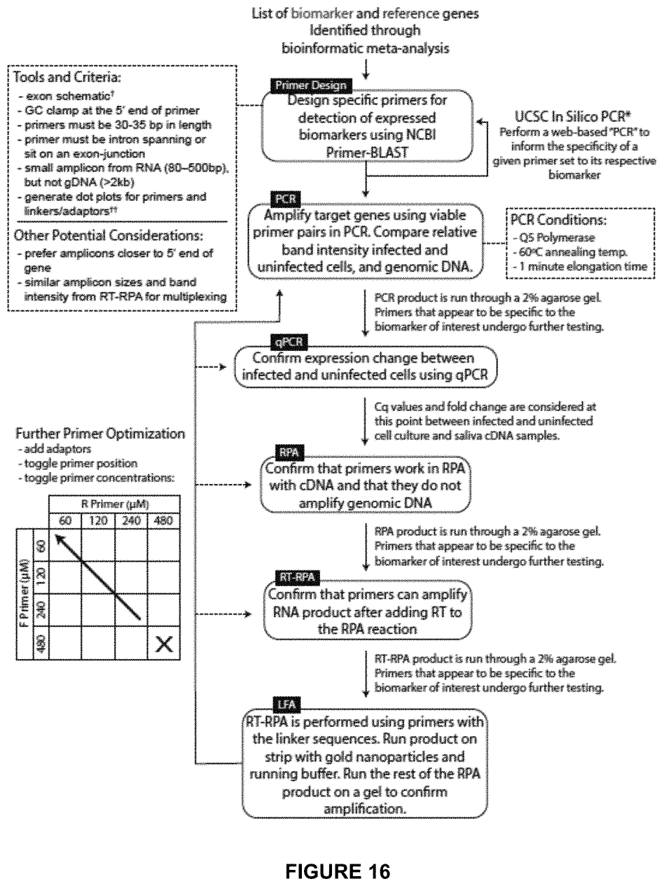

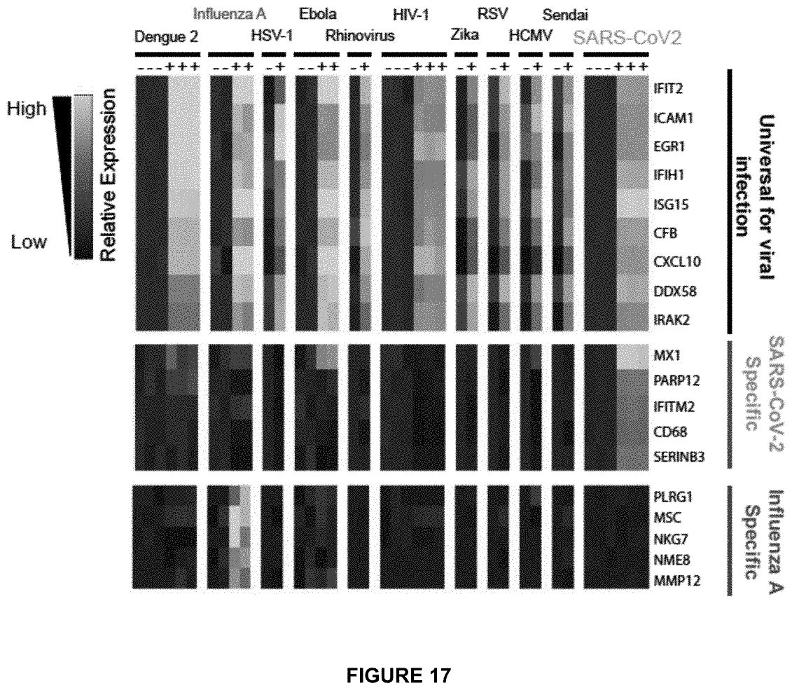

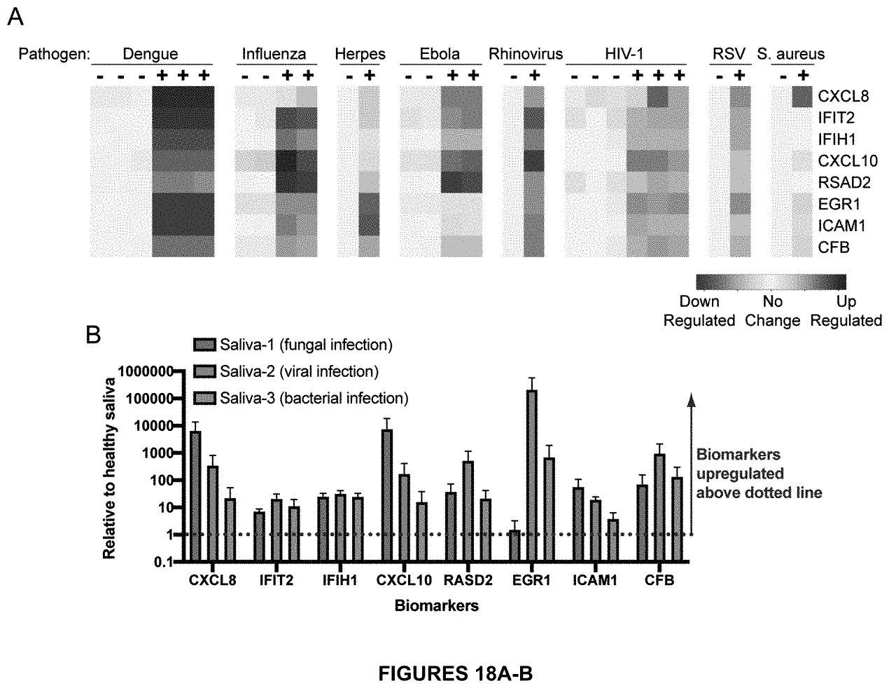

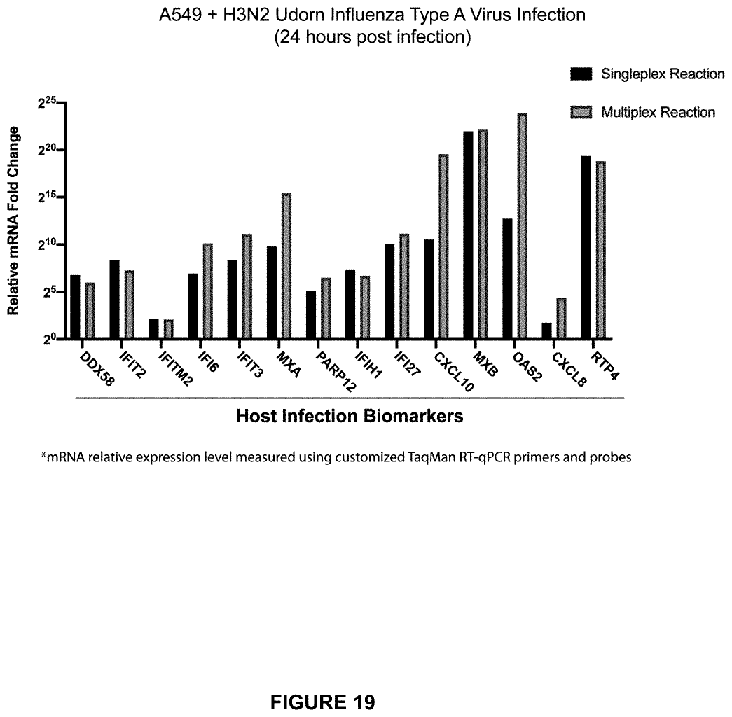

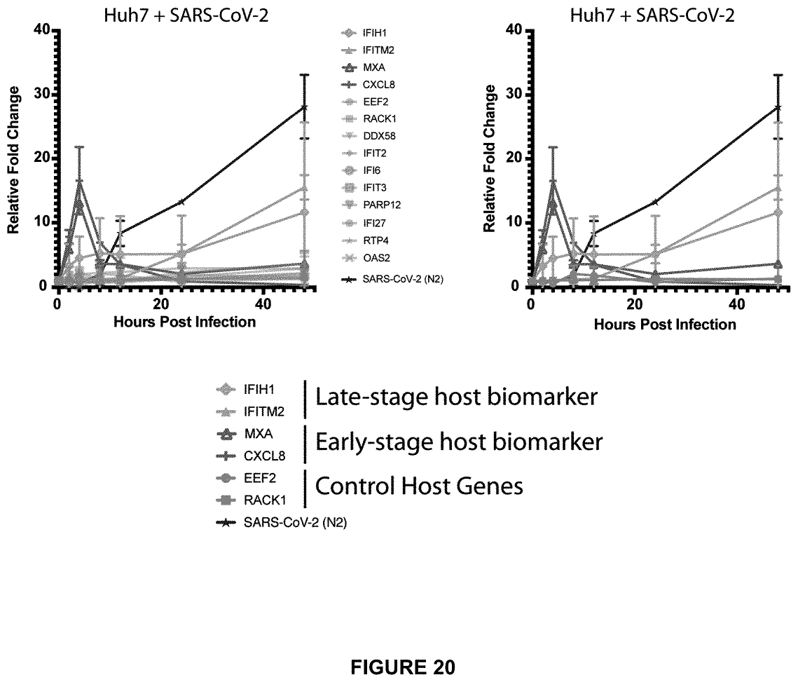

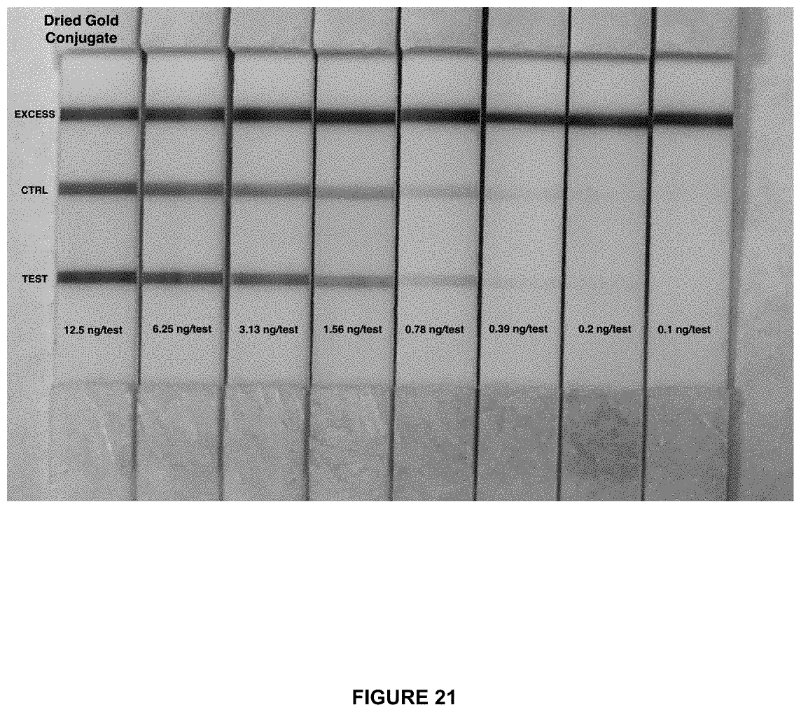

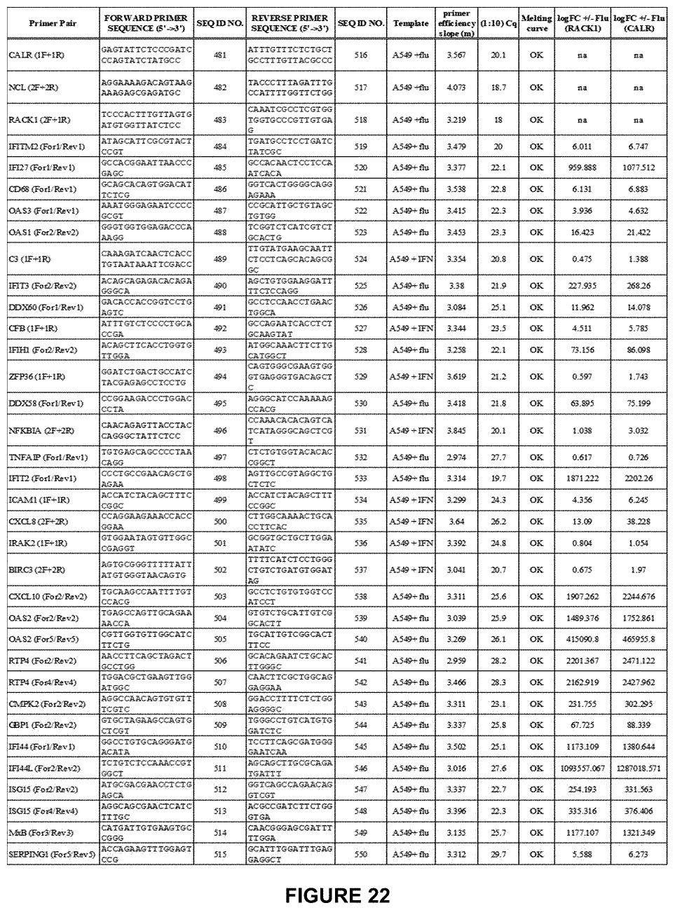

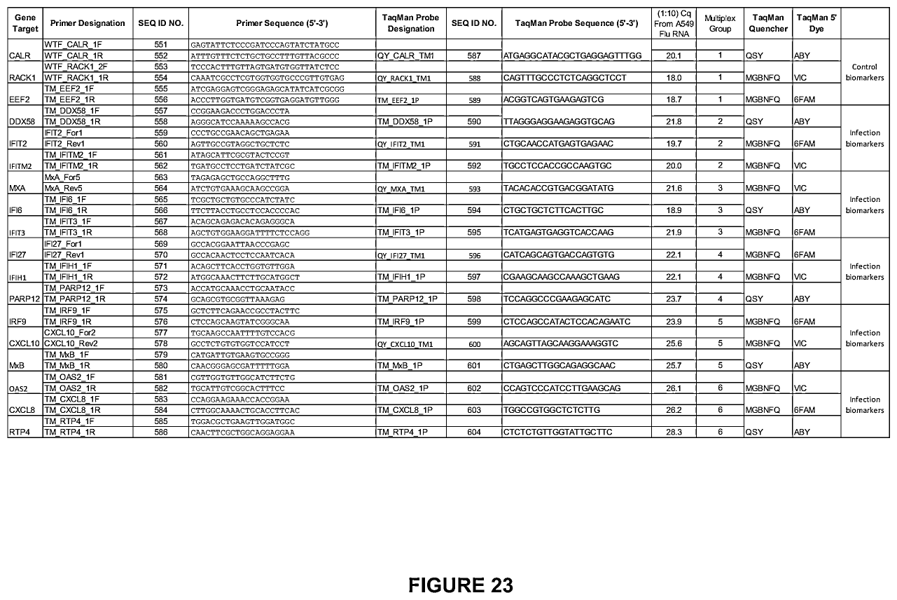

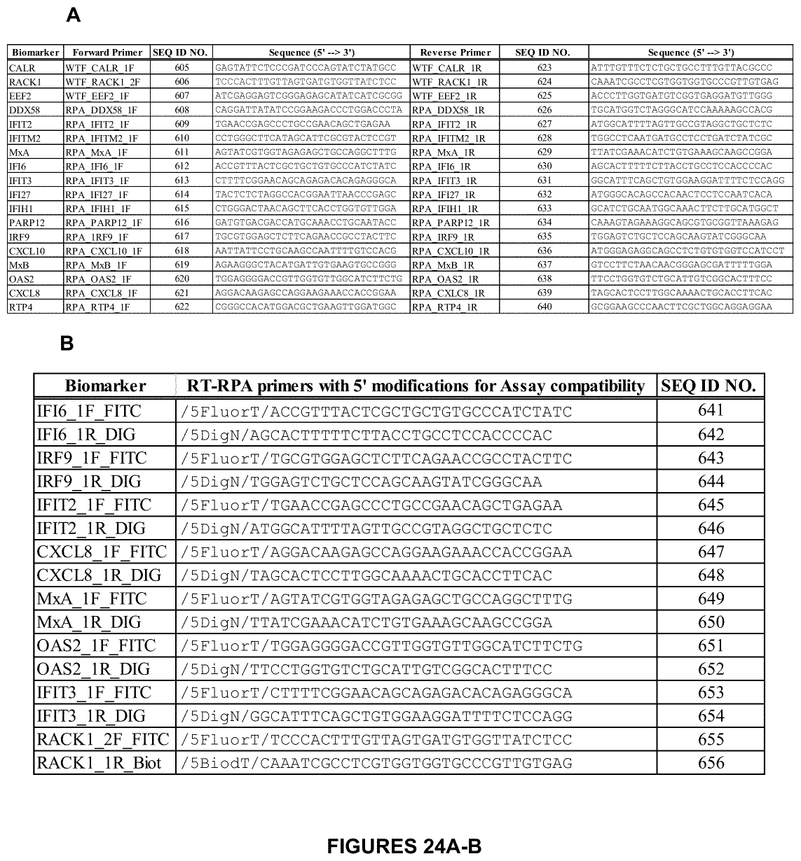

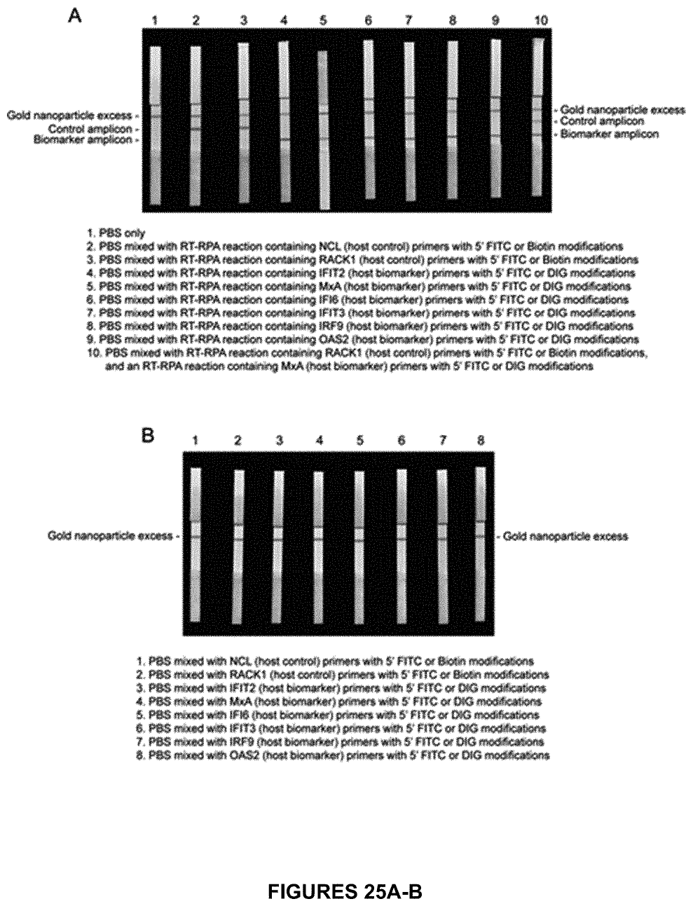

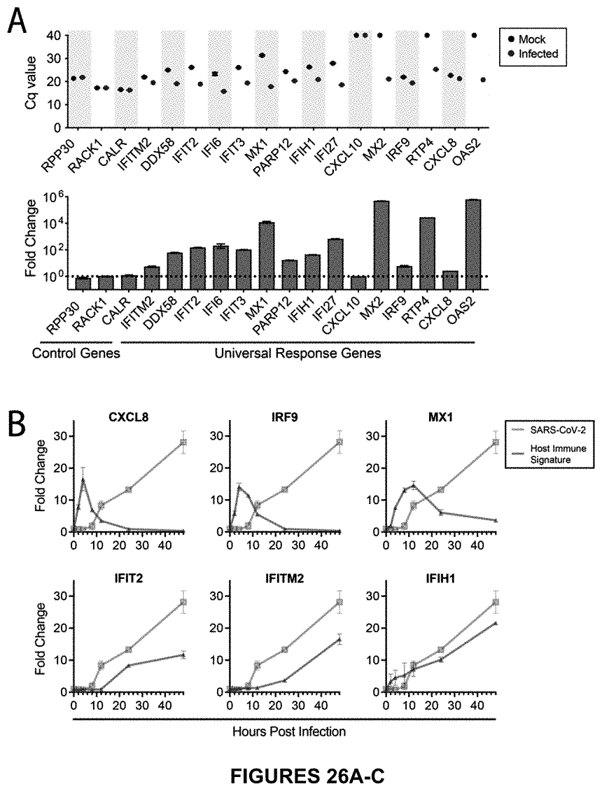

In one preferred embodiment, the inventive technology may provide for the detection of infection of the novel coronavirus SARS-CoV-2 (COVID-19) in a human subject, and in particular host-derived biomarkers of infection generated in response to infection of the novel coronavirus SARS-CoV-2 (COVID-19) in a human subject. As noted above, this example is merely exemplary of a number of different pathogens that may be incorporated in places of the COVID-19 coronavirus. As shown in FIG. 15, in this preferred embodiment, a biological sample, which may preferably include a saliva sample, may be provided by a subject which may contain one or more biomarkers for COVID-19 infection. In this embodiment, a saliva sample, may be further processed, for example by an on-site, or off-site clinical laboratory wherein RNA molecules present in the saliva sample are extracted for further testing. The extracted RNA is then undergoing a qRT-PCR process where the biomarkers of the pathogen, in this case the COVID-19 coronavirus are identified. In the embodiment, one or more of the primer sequencers identified in Table 2 (SEQ ID NO. 469-480) below may be used to identify specific biomarkers produced by the COVID-19 coronavirus. In this embodiment, the subject may provide a plurality of biological samples for RNA extraction and qRT-PCT processing so as to generate a time-course of pathogen biomarkers. For example, as shown in FIG. 15B, multiple samples may provide a quantified baseline progression of pathogen biomarkers from an initial point of exposure to the pathogen.

As noted above the inventive technology may allow the detection of host-derived biomarkers that may be present in a subject's biological sample the virus can be detected before any symptoms of infection may occur. In one preferred embodiment, RNA may be extracted from the biological sample, which in this case is a saliva sample containing host derived biomarkers of infection and further subject to qRT-PCR. In this embodiment, the subject may provide a plurality of biological samples for RNA extraction and qRT-PCT processing so as to generate a time-course of host-derived biomarkers. For example, as shown in FIG. 15B, multiple samples may provide a quantified baseline progression of host-derived biomarkers, such as RNA biomarkers generated by the hosts innate-immune response in response to the COVID-19 pathogen from an initial point of exposure to the pathogen and through the incubation period. Again, as shown in FIG. 15, by combining RNA markers from both the host innate-immune response occurring during the incubation period, and from the COVID-19 coronavirus itself, the invention may expand the detection window for COVID-19 coronavirus infection.

Referring now to FIG. 15C, in another embodiment, a lateral flow assay strip may be configured to detect one or more host-derived biomarkers of COVID-19 infection, and preferably host-derived RNA biomarkers of COVID-19 infection, as well as biomarkers of COVID-19 infection. As noted in the FIG. 15C, the lateral flow assay strip may be configured to include a plurality host-derived RNA biomarkers of COVID-19 infection positioned sequentially according to their prevalence during the time-course of infection established by qRT-PCR described above. In this manner, the lateral flow assay strip of the invention may be able to not only identify a subject that has been exposed to a pathogen, such as the COVID-19 coronavirus, but may include sequential detection lines embedded with one or more biomarkers that correspond to a selected time-course of infection. In this preferred embodiment, a subject may provide a biological sample, and preferably a saliva sample. The saliva sample is allowed to undergo an amplification reaction to increase the quantity of biomarkers and then applied to the lateral flow assay strip as generally described above. In this embodiment, the host-derived RNA biomarkers of COVID-19 infection may be immobilized by target capture probes forming an immobilized aggregate complex which may in turn produce a visible single, again, as generally described above.

Notably, in this embodiment, COVID-19 biomarkers may also be immobilized by target capture probes forming an immobilized aggregate complex which may in turn produce a visible single separate from the host-derived RNA biomarker visual signal. In this manner, a subject, or health care worker may be able to quickly identify: 1) if the subject has been exposed to, in this case the COVID-19 coronavirus; 2) if the subject is infected with the COVID-19 coronavirus but is still in the incubation period of the virus's infection cycle; 3) the approximate time since exposure the COVID-19 coronavirus; 4) the approximate time that the infection with the COVID-19 coronavirus biomarkers may be contagious. As can further be appreciated, in additional embodiment, the lateral flow assay strip may further be configured to identify pre-symptomatic subjects, as well as asymptomatic subjects. Most importantly, the results of the lateral flow assay may allow early identification of infection and facilitate proper quarantine and contact tracing protocols.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Identification of Target Biomarkers of Infection

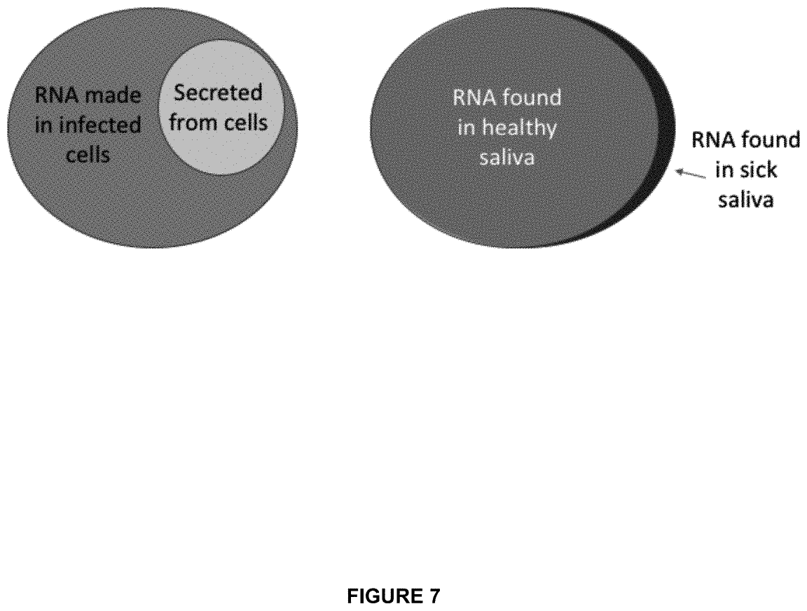

In one embodiment the invention may include systems, methods and compositions for the identification and use of one or more RNA transcript biomarkers. As shown in FIG. 7, in one preferred embodiment, a first tissue culture experiment (left) can be established and tested to identify target RNA transcripts that may be upregulated during an experimental infection, and that may also be secreted from target cells. RNAs that are upregulated may be used as candidate biomarkers and engineered for compatibility with the lateral flow system as generally described above. In parallel, RNAs from healthy and infected human saliva may be characterized in a clinical trial (right) in order to identify RNA biomarkers of infection in humans. Those biomarkers, if not already identified in the tissue culture experiments, will for compatibility with the lateral flow system as generally describe above.

### Example 2: Identification of Early Host Biomarkers

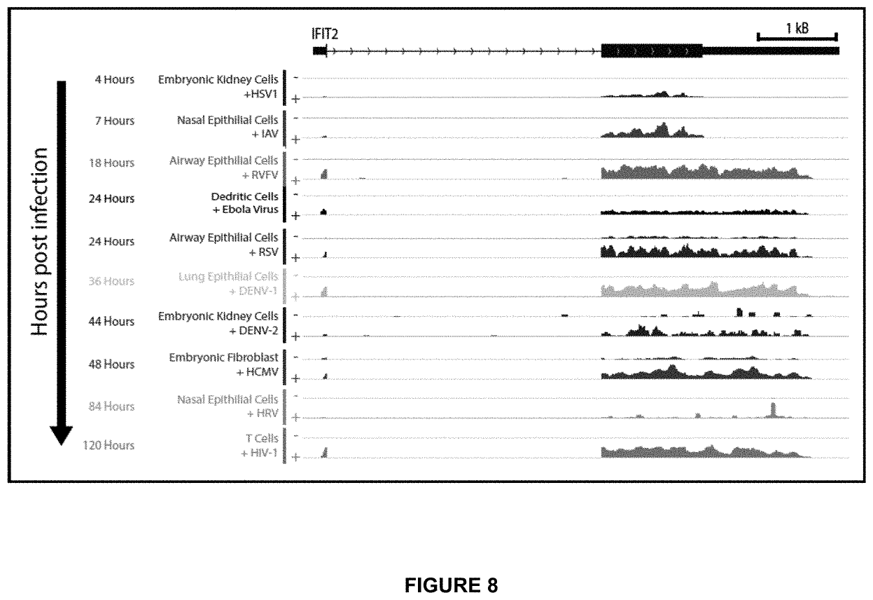

As generally shown in FIG. 8, one embodiment of the invention includes the identification of early host biomarkers for infection using a bioinformatic meta-analysis. In order to identify host nucleic acid biomarkers produced in response to infection at an early stage, the present inventors searched publicly available transcriptomic datasets. The selected datasets were directed to those generated using various human tissue types that are infected by different viruses at multiple time points. The present inventors analyzed these datasets using a standardized bioinformatic pipeline and identified human coding and non-coding RNA that are upregulated in response to infection. These data summarized the host RNA transcripts that are commonly upregulated across different studies. This list of commonly upregulated RNA transcripts was comprised of exemplary candidate RNA transcript biomarkers. The upregulation of these RNA transcripts signals an ongoing infection (Example in FIG. 1).

Concurrently, the present inventors also collected and sequenced RNA purified from saliva samples of healthy and clinical human participant. Through bioinformatic data analysis, the RNA transcripts that are significantly different between healthy participants and infected patients were identified and cataloged. These clinical datasets may then be used to filter out the potential biomarkers. Altogether, the final list of host RNA biomarkers may have the potential to differentiate healthy individuals from subjects that are infected by various pathogens (viruses, bacteria, fungi and protists), using saliva as the non-invasive diagnostic material.

### Example 3: Validation of Target Biomarkers

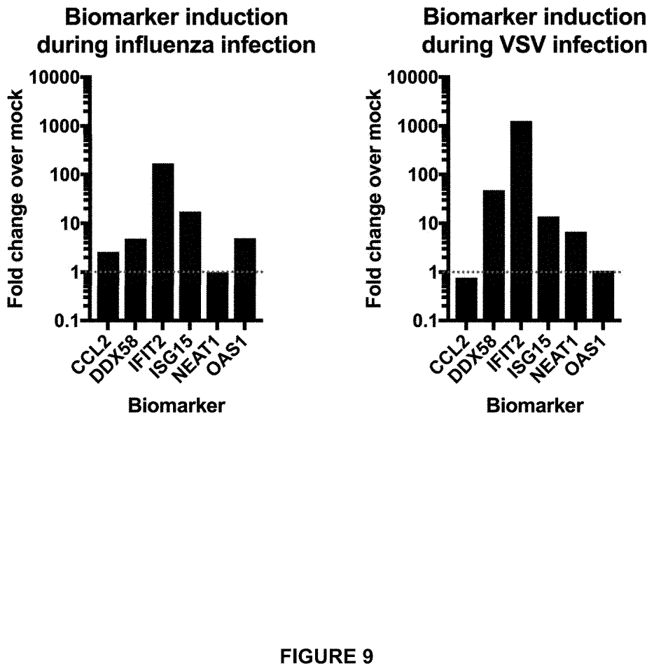

As generally shown in FIG. 9, one embodiment of the invention includes the validation of target biomarkers using quantitative polymerase chain reaction (PCR) protocols. As biomarkers identified using the methods outlined above may be further confirmed in tissue culture infection experiments. Reverse Transcription quantitative PCR (RT-qPCR) of RNA allows specific quantification of the upregulation of candidate biomarkers as a ‘fold change’ in infected cells compared to uninfected cells. Such information helps when evaluating detection sensitivity of the lateral flow assay stick with respect to a given biomarker.

While only six exemplary biomarker candidates are being shown here, such list should not be construed as limiting on the number of biomarkers that may be used with the current invention. Indeed, there may be numerous biomarker candidates that may be incorporated into the invention as described herein.

### Example 4: Isothermal Amplification of Infection Biomarkers from a Bodily Fluid Sample

Upon successful validation of RNA biomarkers that are upregulated during infection in vitro, the target RNA biomarker may be subjected to one or more optimization processes to ensure successful isothermal amplification of the biomarker from human saliva and visualization on a lateral flow assay stick.

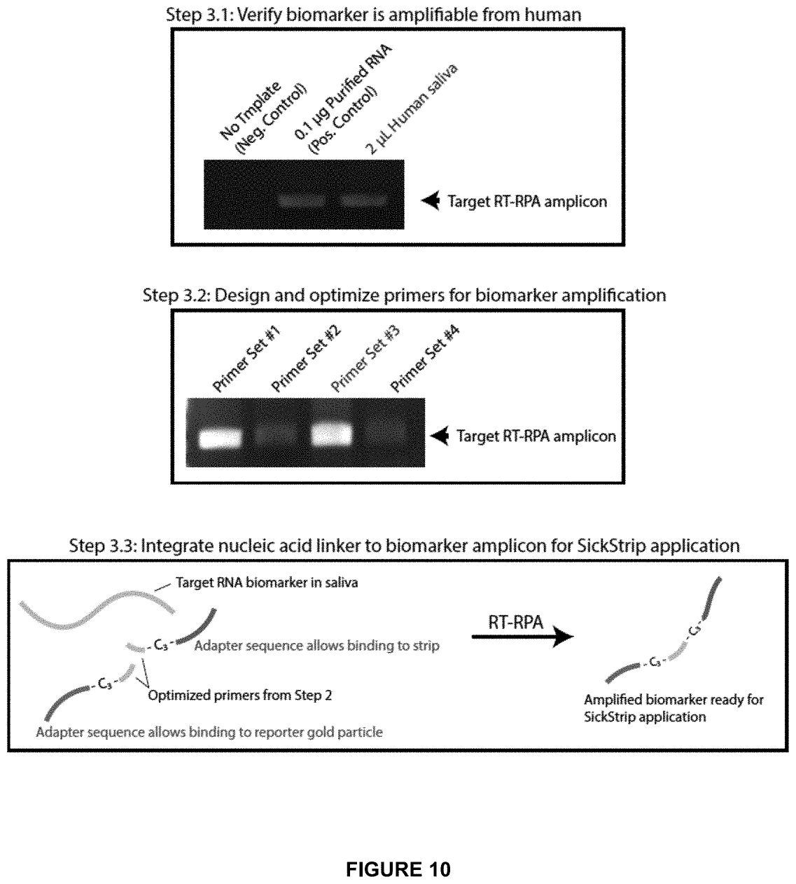

As generally shown in FIG. 10, the presence of a target RNA transcript biomarker in a bodily fluid sample, which in a preferred embodiment may include saliva, is confirmed using an isothermal, one-step reverse transcription and recombinase polymerase amplification (RT-RPA, Piepenburg et al., PLoS Biology 2006) (FIG. 10 Step 3.1). The RT-RPA may be customized by combining TwistDX TwistAmp Basic RPA kit with additional RNase inhibitor, reverse transcriptase and oligo dT primers. The use of this customized reagent allows one-step conversion from target RNA to DNA, which can then be amplified to enhance signal at 37° Celsius (approximate body temperature) within 10-20 minutes.

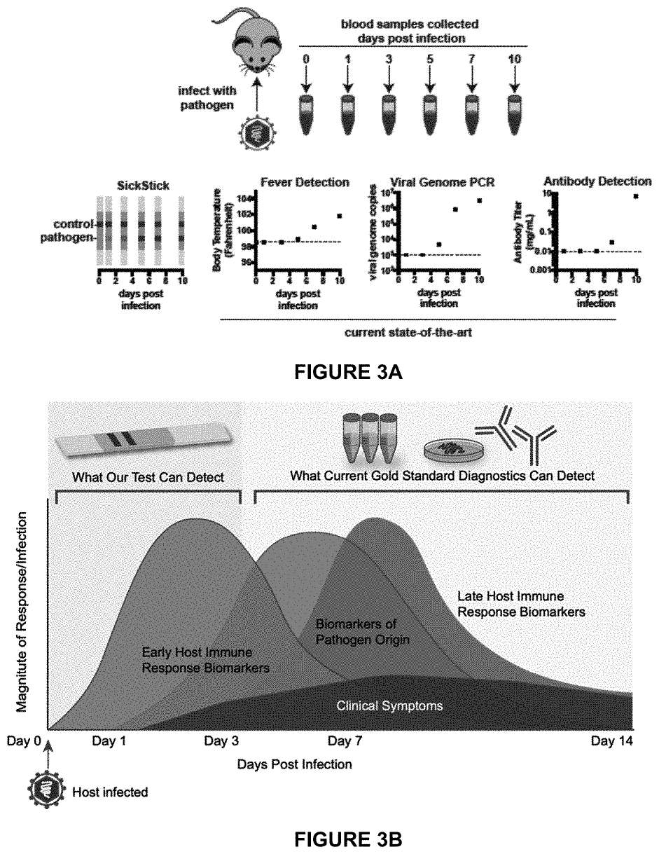

As further shown in FIG. 3 Step 3.1, the amplicon may be separated on 2% agarose gel and visualized by ethidium bromide staining. Comparing to the positive control, the RT-RPA amplified the target RNA biomarker using as low as 2 μL human saliva as input, without additional purification. To achieve efficient amplification and detection, multiple primer sets were designed to amplify the target biomarker (FIG. 10 Step 3.2). These primer sets vary in length and sequence. While keeping other parameters constant, the efficiency for each primer set to amplify the target RNA is compared based on the intensity of amplicon visualized on 2% agarose gel. In the example shown in FIG. 10, while all primer sets where able to amplify the target biomarker, primer set #3 resulted the highest amplification efficiency. Thus, primer set #3 is further integrated into the downstream processes. Finally, based on the test result from Step 3.2, the optimal primer sequences were concatenated with customized adapter sequences on 3′ and 5′ ends that may be complimentary to probe sequences on a gold nanoparticle-based probe and a target capture probe (8) embedded in the test strip, respectively (FIG. 3 Step 3.3). The primers with adapters were then used to amplify the biomarker RNA.

To ensure the adapter sequence remain single-stranded after RPA amplification, the present inventor introduced a tri-carbon chain spacer (C3) within the primer sequence to prevent DNA polymerase from generating the complementary strand of the adapter sequences. As the result, the end product may include an amplified hybrid DNA probe having with a target dsDNA transcript region, while maintaining the single-stranded adapter sequences for downstream hybridization.

### Example 5: Visualization of Amplified Product Using Lateral Flow Assay Stick

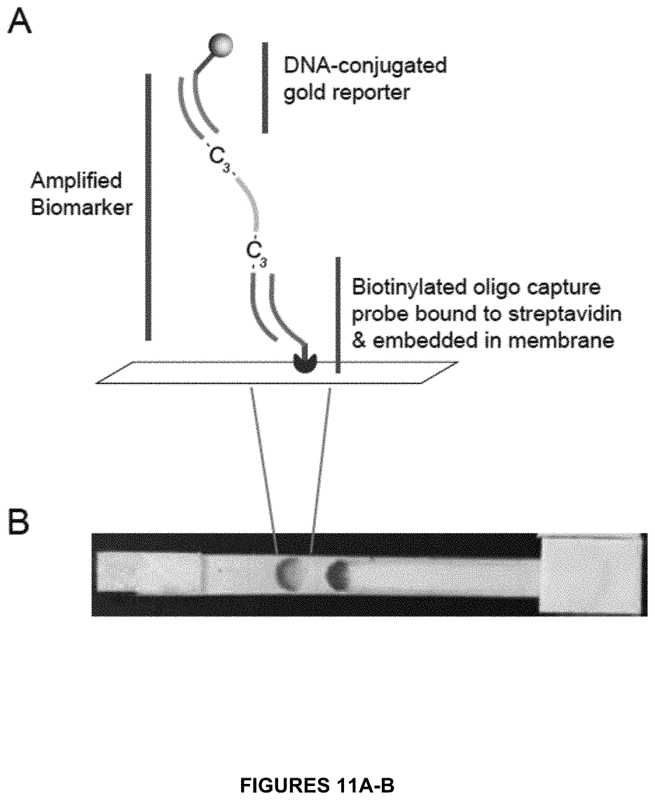

As shown in FIG. 11, the primary unit of the detection assay is a membrane, which is the substrate through which the solution containing the amplified biomarker(s) and the reporter flow. In one preferred embodiment, a membrane (15) may include one or more embedded capture probes (8) that are able to bind complementary probes in the solution that flows through the membrane. As the capture probes bind their respective amplified biomarker or the reporter, a signal appears that indicates infection or no infection. Multiple variables within this broad description of this assay are tunable to be able to express different types of results.

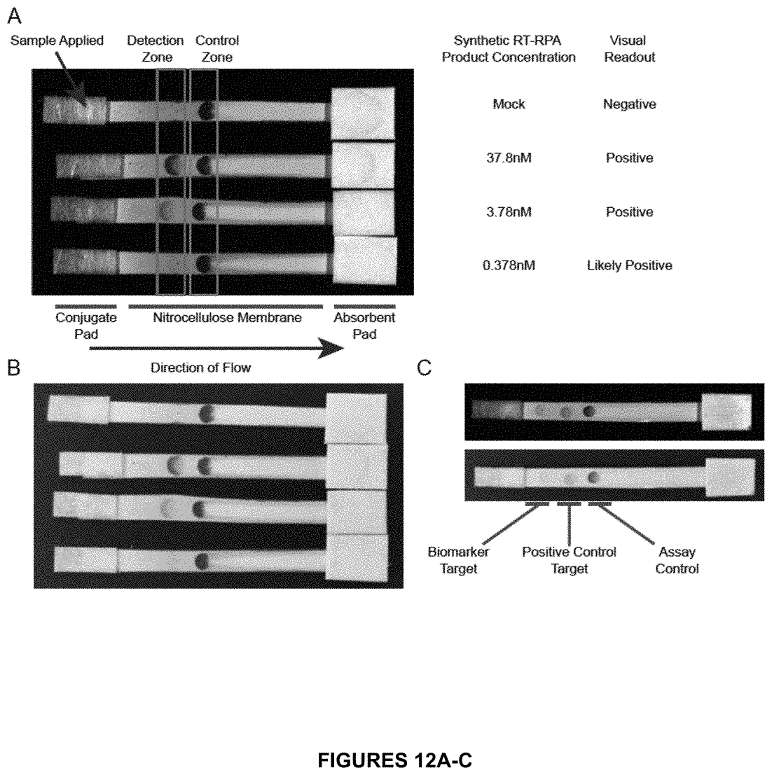

Colorimetric image of a series of test strips run with 10-fold dilutions of a synthetic RT-RPA product are shown in FIG. 12. In this example, a sample contains 2 μL amplified biomarker(s), 10 μL gold reporter, and 8 μL running buffer is applied to the conjugate pad (14) of the test strip (2). (Concentrations of RT-RPA product are listed along with the visual readout.) The solution flows through the nitrocellulose membrane towards the absorbent pad via capillary action. Samples with amplified biomarkers above the limit of detection will aggregate at the first circle in the detection zone. Excess gold reporter that does not interact with amplified biomarkers, either because they were not present in the initial sample or their concentration is below the limit of detection, will continue to flow down the strip and aggregate at the control zone (18).

In the example of the strips shown in (A), a negative result will show one circle on the right side and a positive result will show two circles present (even if faint intensity). To enhance intensity of visual signal, additional 10 μL gold reporter and 8 μL running buffer were combined and applied again to the conjugate pad. (B) Is a color image of the same strips as in (A) shown for comparison. (C) The assay can be assembled to multiplex using different capture probes on the test strip and different adapter primers in the RT-RPA reaction.

### Example 6: Materials and Methods (1)

As shown in the Figures generally, in one embodiment, a lateral flow assay test strip or test strip may be formed of a nitrocellulose membrane which may be a GE Whatman backed nitrocellulose membrane FF120 HP; 5 cm×0.4 cm. A glass fiber conjugate pad may include a Millipore G041 “SureWick” GFCP103000, 1 cm×0.4 cm. A cellulose absorbent pad may include a Millipore C083 “SureWick” cellulose fiber sample pad strips CFSP173000, 1 cm×0.75 cm.

As shown in the figures and described generally above, a conjugated GNP probe may include a biotinylated oligo capture probe bound to streptavidin, which may then be embedded on a nitrocellulose membrane. In one example, 600 μM oligo capture probes were incubated with 200 μM streptavidin for 1 hour at room temperature. With the capture probes now in a complex with streptavidin they may be diluted to a different concentration to optimize binding conditions and signal intensity. In a preferred example, 0.5 μL of solution containing this capture probe-streptavidin complex are pipetted onto nitrocellulose membrane (15) in appropriate orientation, with target probe placed nearest the conjugate pad and control probe placed nearest the absorbent pad. As noted above, a conjugated GNP probe or reporter may be coupled with one or more single-stranded DNA sequences via salt aging method −60 nm or 15 nm or 12.5 nm diameter A running buffer may be mixed with RT-RPA amplified solution product and conjugated gold nanoparticle just prior to running on test strip.

### Example 7: Identification of 69 Human Universal Response Genes

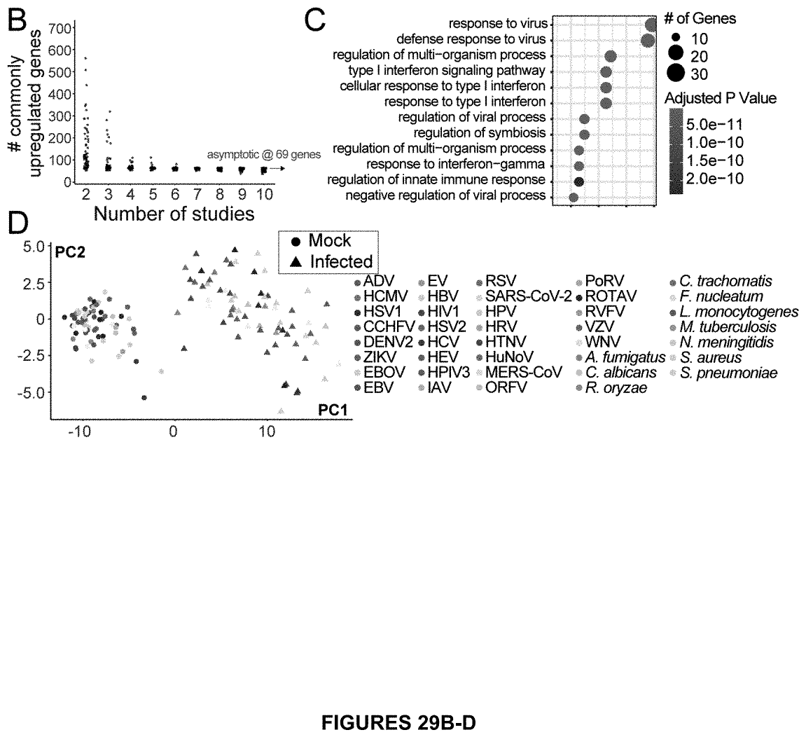

To determine human genes that are commonly upregulated in diverse pathogenic infections, the present inventors first carried out a meta-analysis of publicly available data. We obtained a total of 71 relevant datasets, all profiling in vitro transcriptional responses of cultured human cells infected with a variety of pathogens (28 viral, 7 bacterial and 3 fungal pathogens, with many pathogens represented by more than one dataset; Table 3). Each study includes paired transcript sequencing for infected and mock-infected human cells, usually in multiple replicates. For each dataset, raw RNA sequencing reads were retrieved from the NCBI short-read archive and analyzed as described herein. Despite the many variables in these datasets (pathogens, human cell lines, labs conducting the studies), the present inventors obtained a list of 69 genes that are consistently upregulated in infected cells across the array of pathogen types tested (FIG. 29A and genes are listed in Table 4). We refer to these as “universal response” genes. Importantly, the number of universal response genes reaches an asymptote of 69 genes as more and more studies were added to the analysis (plotted up to 10 studies in FIG. 29B). After reaching 69 genes, the addition of more datasets did not change the constitution of this group of 69; new genes were no longer added or subtracted from the set as datasets accumulated. Therefore, while some aspects of the transcriptional response to infection are specific to certain classes of pathogens, these 69 genes represent a core universal response to infection.

Consistent with our understanding of innate immunity, universal response genes mainly belong to pathways related to cellular antiviral functions and type-I interferon responses (FIG. 29C). We then carried out principal component analysis of all the datasets used using the expression profiles of these 69 genes (FIG. 29D). Of the many variables involved, the main contributor to the data variance (PC1; which explains 81.6% of the variance) separates these in vitro experiments by conditions of infected (triangles) or uninfected (circles). This suggested that levels of mRNAs from this group of 69 genes can differentiate infected from uninfected human cells.

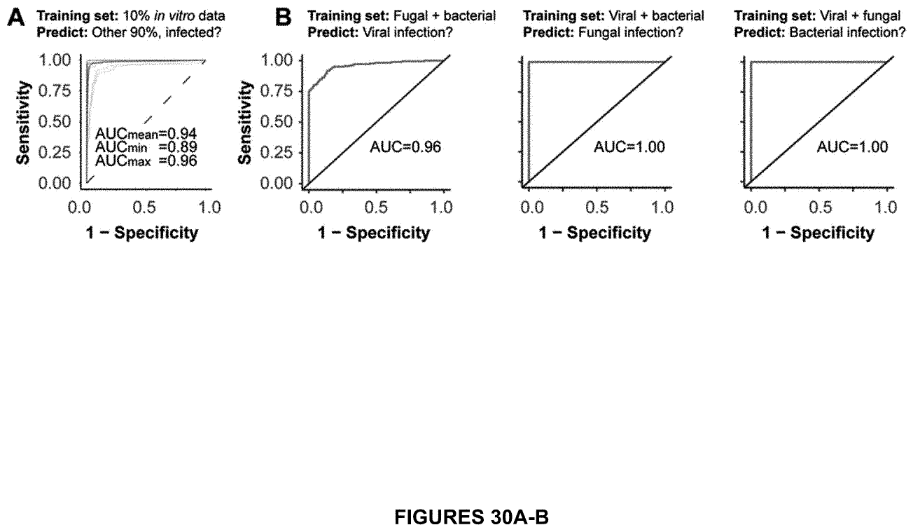

We the present inventors assessed whether the abundance of these mRNAs in blinded human tissue culture samples could predict whether they had been infected or not. Using the 387 samples (meaning, independent experimental replicates) represented in the 71 in vitro infection datasets, we carried out cross-validation using a linear regression model. Specifically, we first established the linear regression classifier using the expression data of the 69 genes in 10% of the samples, randomly selected. Next, we evaluated the predictive power of this model to classify the remaining 90% of the samples as infected or not. This cross validation was repeated 10 times, and the accuracy of classification is summarized via receiver operating characteristic (ROC) curve (FIG. 30A). Overall, the cross validation resulted in a mean area under the curve (AUC) of 0.94, also interpreted as a 94% chance of distinguishing mock from infected conditions. The worst outcome of the 10 repeats had an AUC of 0.89, and the best an AUC of 0.96.

We then performed additional cross validation analyses among different types of infections (FIG. 30B). We trained the classifier using only viral and bacterial samples and then classified the fungal samples as infected or not. This was highly successful and yielded a ROC cure with an AUC of 1.0. We then trained the classifier using only viral and fungal samples and then classified the bacterial samples as infected or not (again, AUC of 1.0). Finally, we trained the classifier using a combination of bacterial and fungal samples, and this model classified the viral samples as infected or not with AUC of 0.96. Collectively, this indicates the upregulation of these universal response in human cell lines can correctly identify infection status, independent of the cell and pathogen types involved, and that these 69 genes truly represent a universal response to infection.

### Example 8: Host Immune Signatures are Consistently Upregulated in Infected Human Saliva

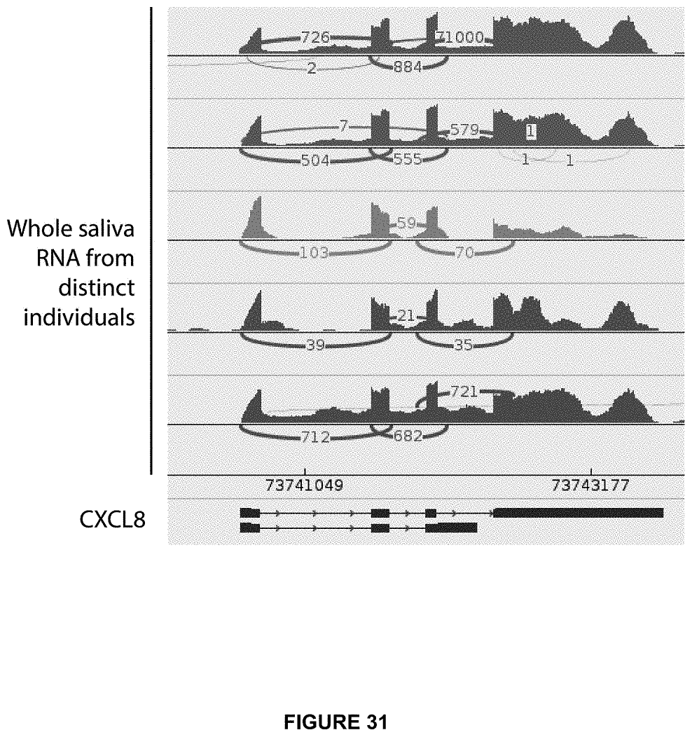

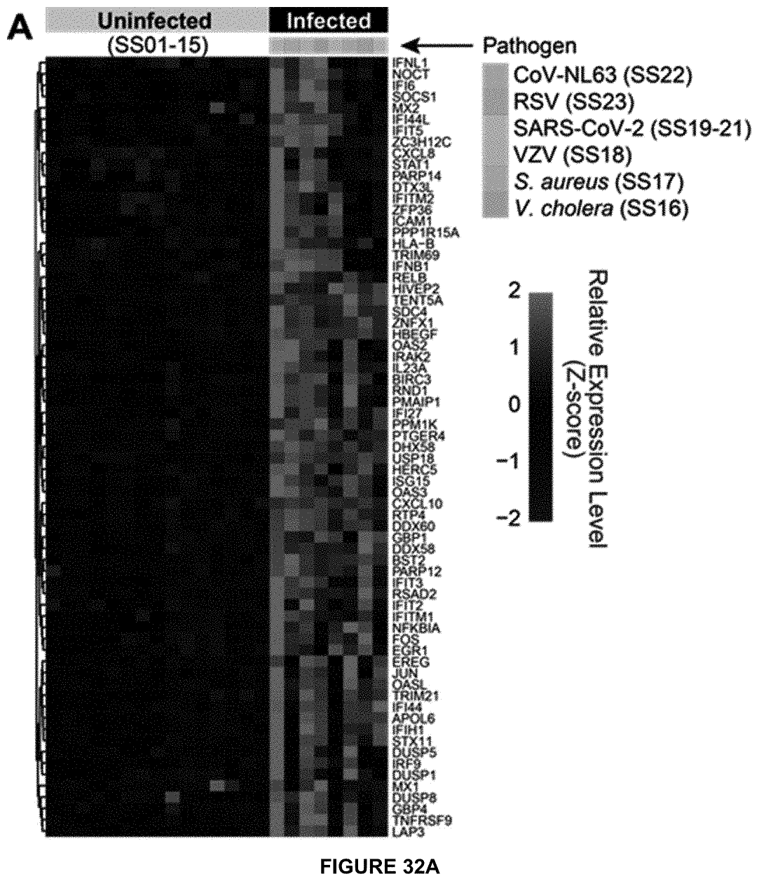

The present inventors next evaluated the abundance of mRNAs from these 69 genes could classify humans as infected or not. We obtained saliva samples from 15 healthy individuals and from 8 infected individuals. Of the latter, six saliva samples are from patients in our infectious disease clinic (Table 5). Three had been diagnosed with SARS-CoV-2 (enrollees SS19-SS21), one with Vibrio cholera (SS16), one with Staphylococcus aureus (SS17), and one with varicella-zoster virus (VZV; SS18). Two additional saliva samples were included from apparently healthy individuals from whose saliva we were able to map reads to pathogen genomes (SS22, CoV-NL63 seasonal coronavirus; SS23, respiratory syncytial virus (RSV)) (see Methods). Collectively, these eight enrollees represent six respiratory tract infections caused by RNA viruses, one infection caused by a DNA virus (VZV), and two bacterial infections. Total RNA was prepared from each of these 23 human saliva samples, followed by depletion of bacterial and human ribosomal RNA. RNA with high integrity can be readily isolated from saliva (FIG. 31). Libraries were sequenced with high-throughput short-read sequencing.

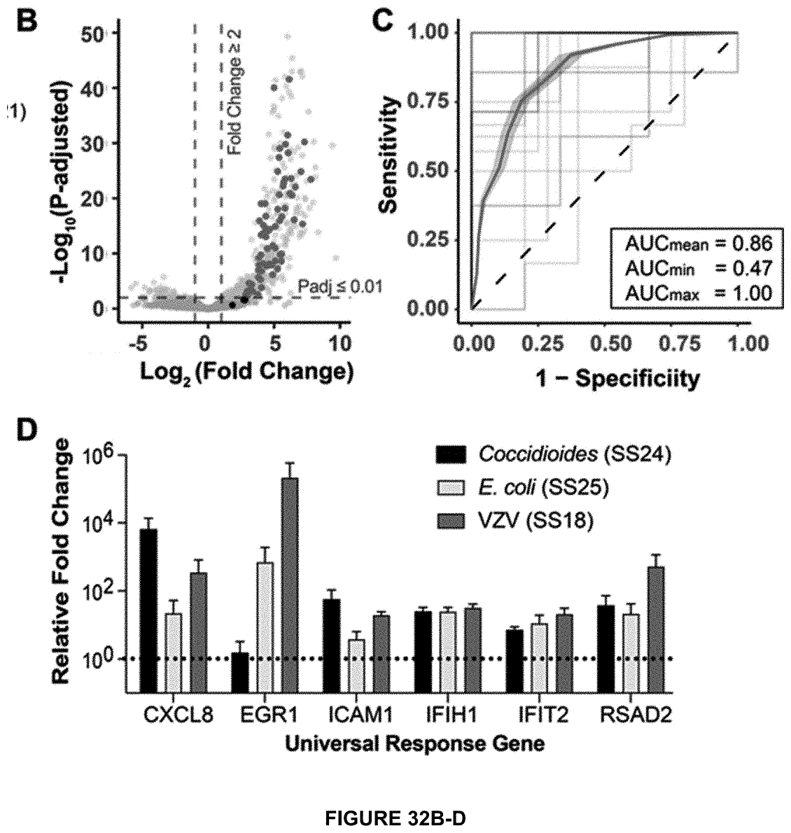

Consistent with the in vitro meta-analysis, 66 out of the 69 human universal response gene transcripts were significantly enriched in the saliva of all 8 infected individuals compared to healthy individuals (FIG. 32A). This confirms that mRNAs identified as being significantly upregulated in a diverse panel of in vitro tissue culture infections are also upregulated in saliva of infected individuals. In total, there were 544 genes that were significantly upregulated across all the infected individuals (light pink dots in FIG. 32B, adjusted P-value≤0.01, Fold Change≥2; Table S4). Of these, the universal response genes are shown as dark red dots and are not necessarily the most highly up-regulated transcripts. We next carried out cross validation and found that a classifier trained on the mRNA levels of universal response genes in the 71 in vitro datasets analyzed above can correctly classify our human saliva samples as having come from someone who is infected or healthy, just from the abundances of these mRNAs in their saliva (FIG. 32C, Mean AUC=0.86). Thus, this classification was made correctly 86% of the time.

The present inventors next verified this finding with RT-qPCR and were able to include two additional patient samples for this analysis. The new saliva samples come from an enrollee being treated for a Coccidioides fungal infection (SS24, Table 5) and another enrollee being treated for Escherichia coli bacterial infection (SS25, Table 5). We amplified mRNA from six of the universal response genes (CXCL8, EGR1, ICAM1, IFIH1, IFIT2, RDAS2) from the saliva from these additional enrollees, and from SS18 (Table 5), a patient being treated for VZV (viral) infection. We observed from 10- to 10⁵-fold upregulation of most of the host mRNAs within the saliva of infected individuals compared to healthy ones (FIG. 32D). Importantly, the infected individuals analyzed thus far have carried pathogens known to have different primary replication sites, including respiratory tract (RSV, CoV-NL63, SARS-CoV-2, and Coccidioides), digestive tract (V. cholerae and E. coli), and pulmonary tract (S. aureus), yet these signatures are reliably detectable in saliva.

### Example 9: Development of a Multiplex TaqMan RT-qPCR Assay to Monitor mRNAs Derived from Universal Response Genes

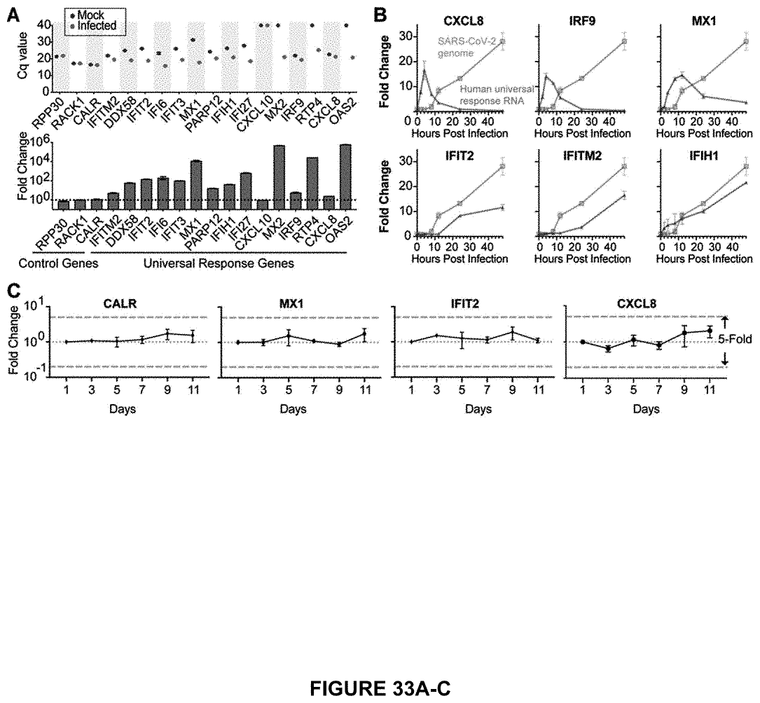

To measure the transcription levels of the universal response genes more efficiently and quantitatively, we moved away from total RNA sequencing and developed a multiplex TaqMan RT-qPCR assay that measures the level of mRNA produced from 15 of the 69 universal response genes. Together with 3 internal controls genes (RPP30, RACK1, and CALR), the levels of all 18 genes are measured in a total of 6 multiplexed reactions. We optimized this TaqMan assay on RNA harvested from A549 human lung cells mock infected, or infected with influenza A virus (H3N2/Udorn) at MOI of 0.1 for 24 hours. Using these samples, we confirmed that the assay can measure each mRNA over a large dynamic range (Ct 15-40) with small amount of input RNA (≥100 ng) (FIG. 33A). At this high MOI but relatively short infection timepoint, already 14 out of the 15 measured genes are upregulated. The range of mRNA upregulation in infected cells ranged from 2.6-fold (CXCL8) to 6.1×10⁵-fold (OAS2). Because this experiment measured the abundance of host mRNAs at a single timepoint of a synchronized infection, we next infected Huh7 human liver cells with SARS-CoV-2 and collected cells on a time course. The kinetics of expression of six of the universal response transcripts is shown in FIG. 33B. Some universal response genes (CXCL8, MX1, and IRF9) are upregulated in the early time points of the infection but are then rapidly downregulated within the first 24 hours, whereas the upregulation of other genes (such as the classical type-I interferon inducible genes, IFIT2, IFITM2, and IFIH1), tracks with viral genome replication. This result suggests that the abundance of mRNA from any particular gene will depend on the timepoint during infection, at least in a synchronized infection taking place in a tissue culture dish.

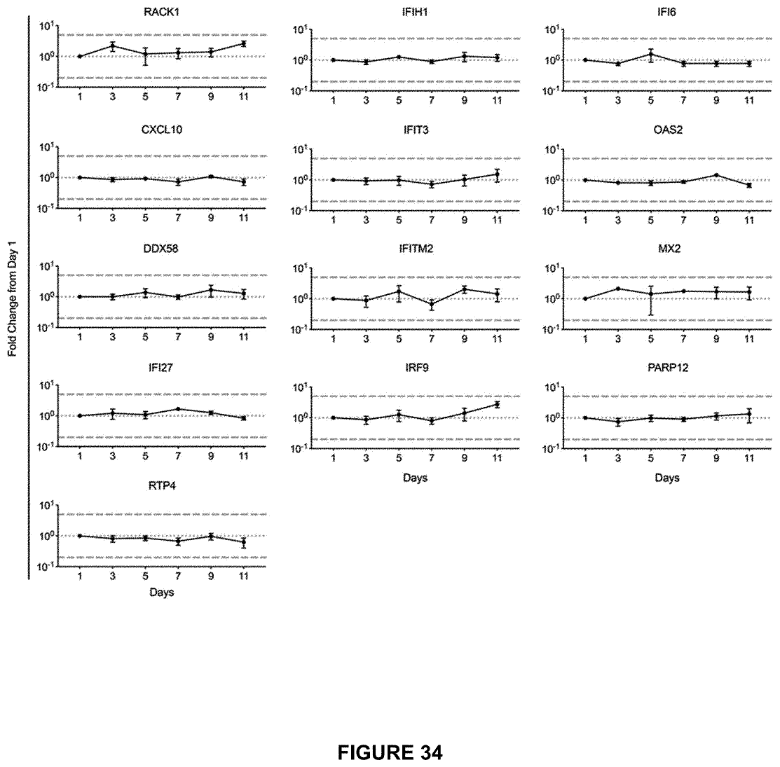

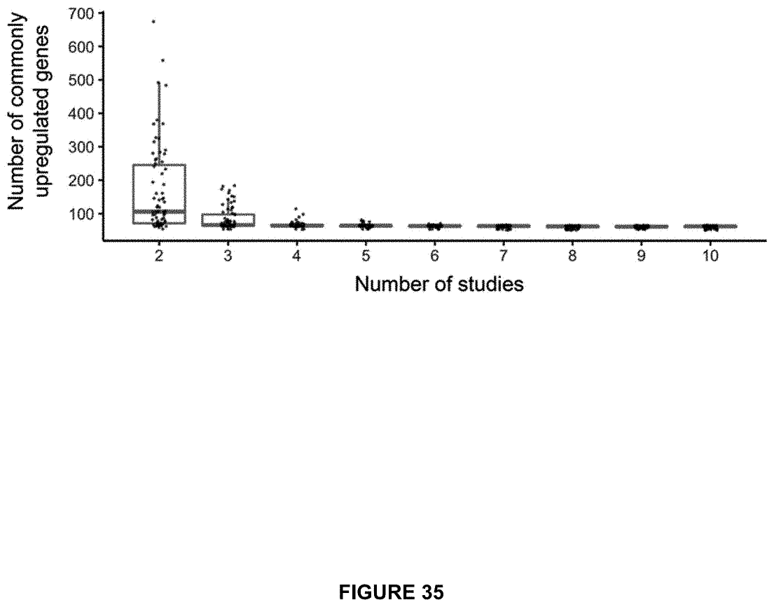

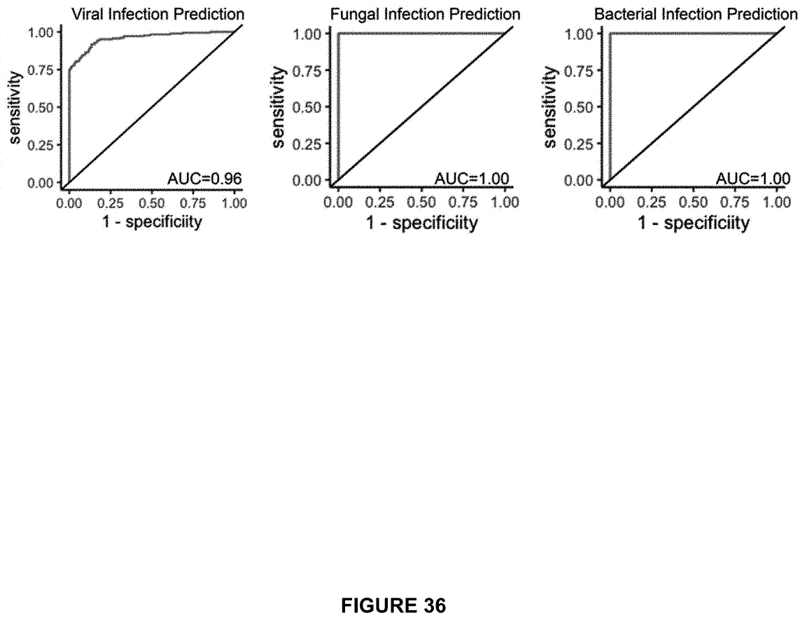

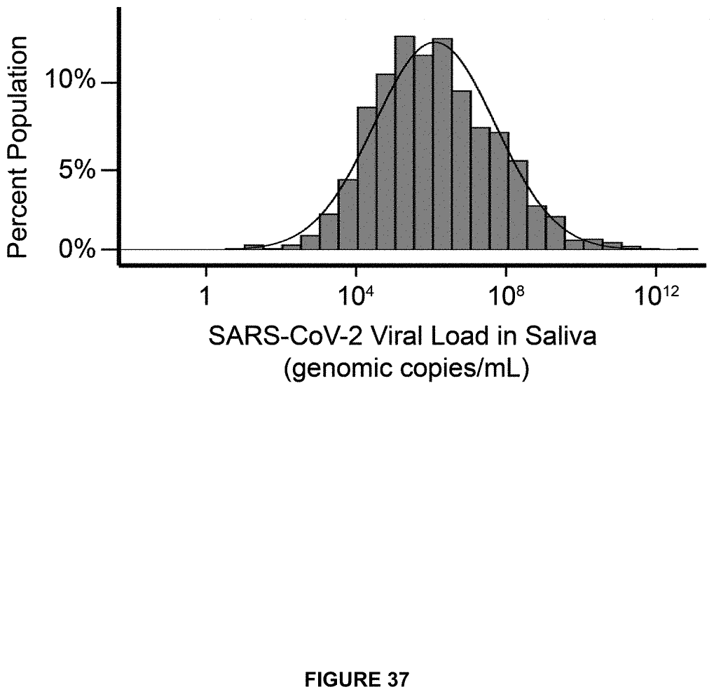

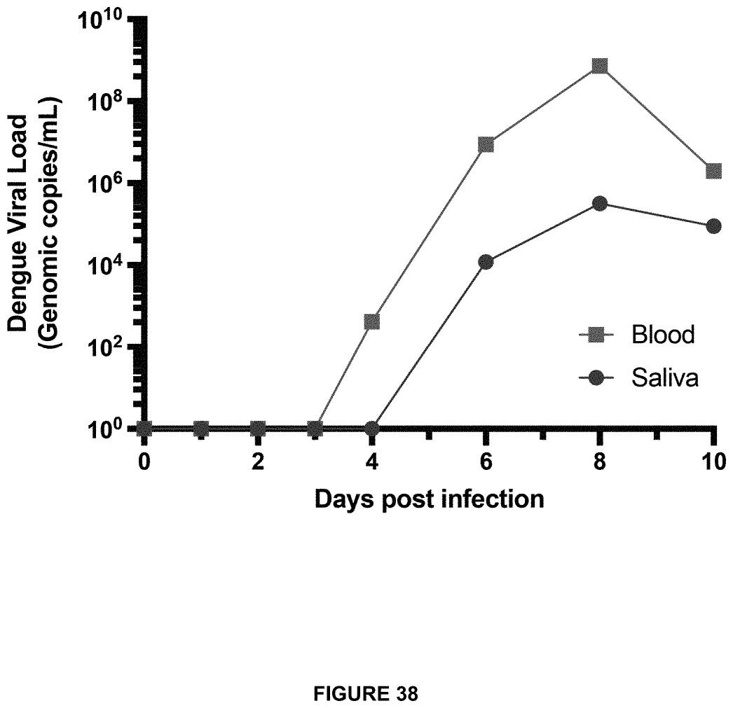

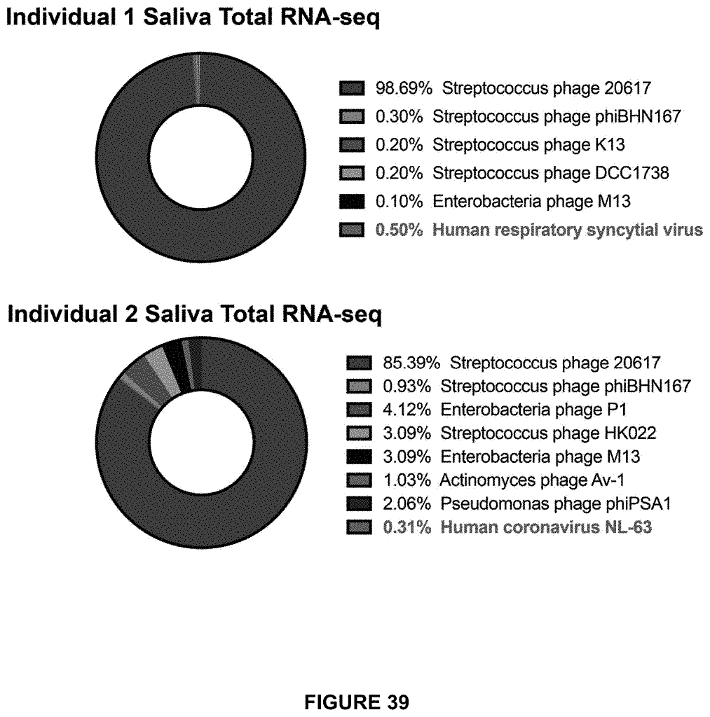

The present inventors next sought to determine if the mRNA levels of universal response genes also vary over time in human saliva. We enrolled 7 apparently healthy individuals who were asked to collect saliva samples daily over a period of 11 days (FIGS. 33C, 34). When RNA from these saliva samples was analyzed with the multiplex TaqMan assay (Methods), the expression level of the universal response genes remained relatively stable overtime. When compared to day 1, transcript abundance in saliva changed no more than 5-fold in subsequent days. Together, the multiplex TaqMan RT-qPCR assay described herein can be used to reliably determine the relative abundance of these universal response gene transcripts from in vitro infections and human saliva samples alike. An interesting but unresolved issue requiring longitudinal studies is how the expression of these universal response mRNAs would change over time during a human infection.

### Example 10: Universal Response Transcripts in Saliva can Detect Infection in Asymptomatic SARS-CoV-2 Carriers

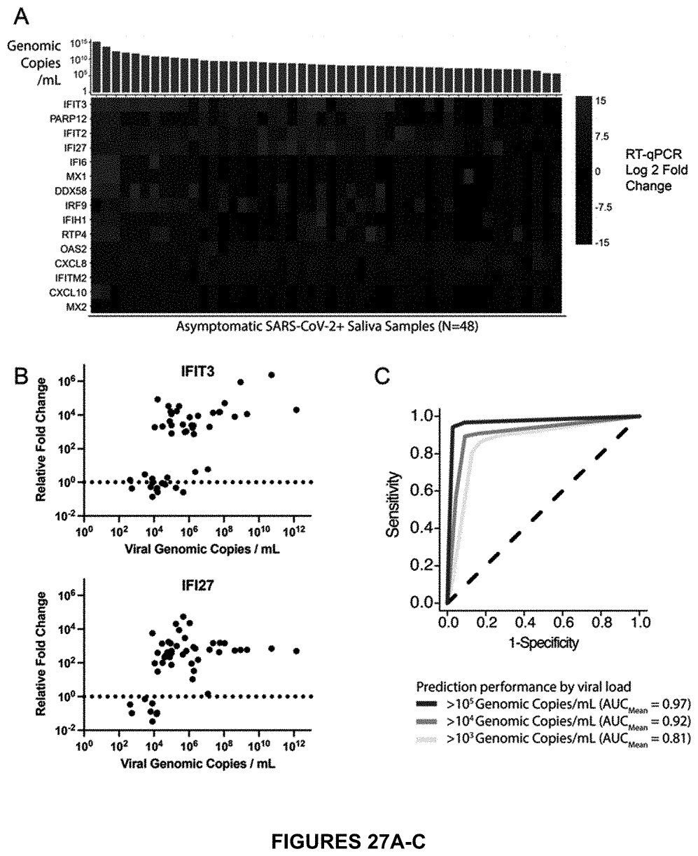

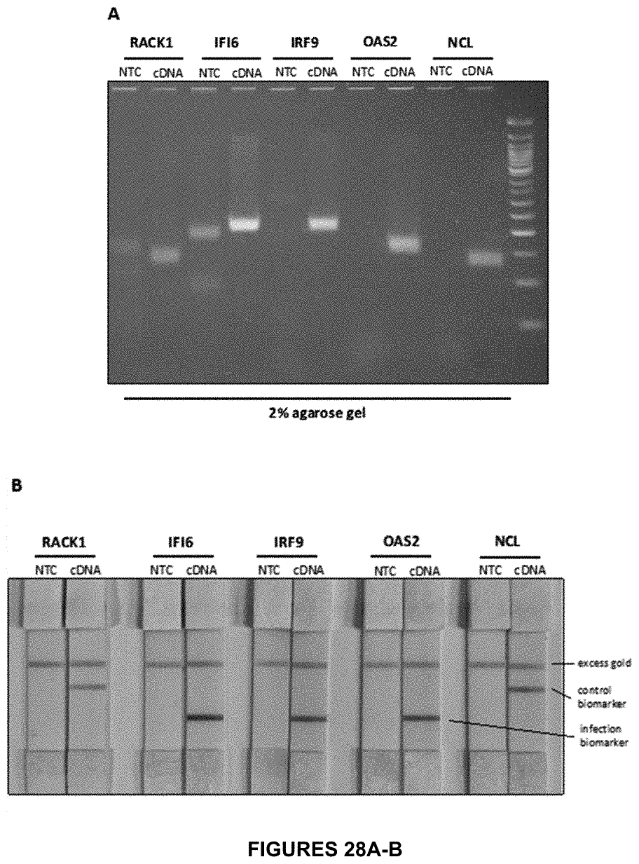

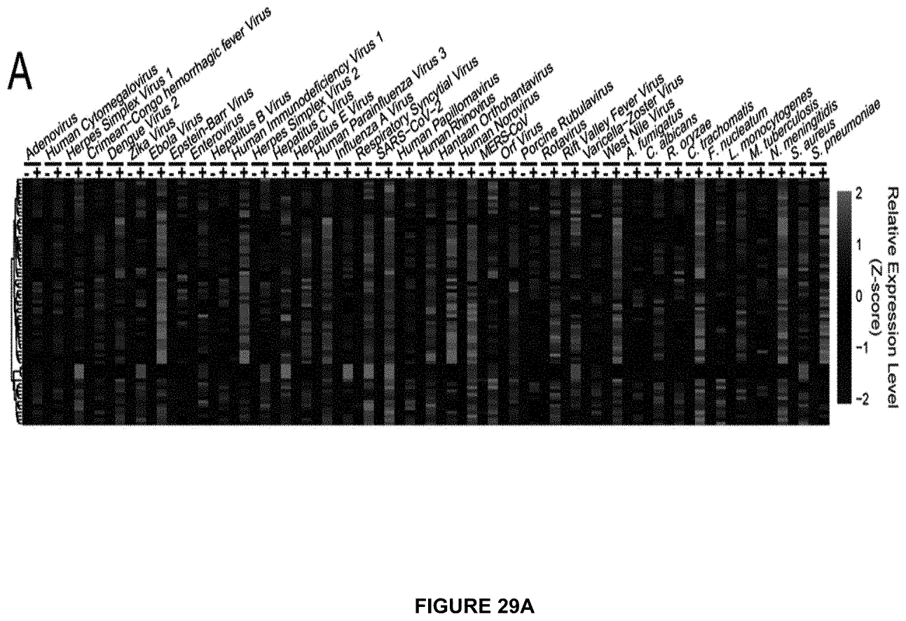

The present inventors next sought to determine if universal response mRNAs in saliva can identify infection, even in individuals with no symptoms. During the 2020-21 academic year, the University of Colorado Boulder carried out weekly SARS-CoV-2 screening for students and staff. The screening effort enabled us to enroll university affiliates into an associated human study. All saliva samples were screened for SARS-CoV-2 by a RT-qPCR test. Enrollees were asked to confirm the absence of any symptoms at the time of saliva donation. We examined the levels of mRNA from universal response genes in the saliva of 48 SARS-CoV-2 positive saliva and 20 non-infected individuals (FIG. 27A). We observed higher levels of universal response mRNAs in the saliva of most of the SARS-CoV-2 positive individuals. Importantly, we noticed strong correlation between the level of mRNA observed and saliva viral load. Within saliva samples that carried significant viral load, almost all had elevated level of mRNAs deriving from universal response genes.

The correlation between viral loads and the expression of the universal response genes is highlighted by further analysis. Specifically, for two of the universal response genes (IFIT3 and IFI27), we plotted the relative fold change of mRNA in saliva against the number of viral genome copies in saliva (FIG. 27B). For SARS-CoV-2, infectious virions are almost never recovered from individuals with a viral copies measurement less than 10⁶ copies per mL. Individuals with lower viral copies/mL are either on the rapid progression to high virus titers at the beginning of infection, or on the long slow tail of recovery after infection. Interestingly, the mRNAs of IFIT3 and IFI27 accumulate in saliva very near this point, at the transition of viral titers to above 10⁴-10⁶ viral copies/mL. This is consistent with a model where mRNAs from universal response genes accumulate in saliva specifically during periods of acute viral replication.

To evaluate the accuracy of using universal response mRNA abundance in saliva to distinguish infected from non-infected humans, we carried out cross-validation using linear regression models established on half of the data from our human studies (N=34). This classifier was then used to classify all remaining human saliva samples as infected or not (N=34, FIG. 27C). Overall, this analysis resulted in an area under curve (AUC) of 0.92 and 0.97 for classifying infection status for individuals with viral load greater than 10⁴ genomic copies/mL and 10⁵ genomic copies/mL, respectively. The evaluation again supports that the abundances of mRNAs from universal response genes, detectable in saliva, are highly reliable in predicting whether or not an individual is infected. This is especially true for individuals harboring viral loads consistent with the infectious phase of disease. Importantly, none of these individuals reported symptoms at the time of their saliva being collected, suggesting that the mRNAs in saliva have more predictive power over infection than self-reported symptoms.

### Example 11: Materials and Methods (2)

Meta-Analysis of NCBI SRA Transcriptomics Datasets:

We carried out meta-analysis of RNA-seq datasets publicly available at the NCBI SRA database. Our criteria for choosing datasets where that human cells in culture were infected with a bacterial, viral, or fungal pathogen, and then the cellular transcriptome was sequenced along with that in a mock-infected control. We obtained a total of 71 relevant in vitro infection datasets. From these datasets, raw RNA sequencing reads in FASTQ format were downloaded, trimmed using BBDuk (BBMap v38.05)⁴⁹ and mapped using HISAT2 v2.1.0⁵⁰ to human genome assembly hg38. Using NCBI RefSeq genome annotation, we then counted the mapped reads assigned to gene or transcripts using FeatureCount (Subread v1.6.2)⁵¹.

First, we looked for genes that were upregulated in each infected dataset versus its matched mock infection. For each individual dataset, the infected replicates were compared to the corresponding mock replicates via the DESeq2 Wald test (v3.1.3)⁵², from which the fold change and Benjamini-Hochberg adjusted p-values were obtained. Correction for multiple testing was performed throughout. Next, we looked for the subset of these genes that was statistically enriched in infected datasets overall. DESeq2 results from individual datasets were ranked and combined based on the magnitude and consistency of upregulation across the datasets. Specifically, the gene rank, r_{g} is assigned to each individual dataset following the formula:

r_{g}=Rank(−log 10(Pval_{Adj})×fold change)

Next, to determine which gene is consistently upregulated across different studies, the rank is combined via rank sum statistics. With n studies, the rank sum for each gene, g, is calculated as:

RS_{g}=(Σᵢr_{g,i})

Hence, each gene is sorted based on the RS_{g}. We then filtered the gene list based on the within-study adjusted p-value and required that the gene to be significant (p_{adj}<0.05) in 90% of the datasets. As the result, we obtained 69 universal response genes ranked by the statistical significance comparing infected vs. mock groups and by the consistency across datasets.

Human Saliva Sample Collection, Handling, and RNA Preparation:

Samples SS4, SSS, SS12-SS21, SS24 and SS25 were collected under protocol 17-0562 (U. Colorado Anschutz Medical School; PI Poeschla), where adult participants were consented verbally and donated up to 5 mL of whole saliva and/or 50 mL whole blood per visit with no more than two visits per week and no more than 500 mL blood volume drawn per patient. Saliva was collected into Oragene saliva collection kit (DNA Genotek CP-100). The saliva is mixed with the stabilization solution in the collection kit and stored at room temperature for no longer than 2 weeks before being processed for RNA purification. Blood collected from patients with confirmed or suspected infection did not exceed the lesser of 50 mL or 3 mL per kilogram in an eight-week period. Diagnosis of these individuals was provided in the form of clinical notes.

Saliva samples from individuals SS1-SS3, SS6-SS11, SS22, and SS23 were collected under protocol 19-0696 (U. Colorado Boulder, PI Sawyer), where anonymous adults verbally consented and donated up to 2 mL of whole saliva. Saliva was collected into Oragene saliva collection kit as mentioned above. For these individuals, infection status was later determined by in silico metagenomic detection using GOTTCHA (v1.0b)⁵³ using the RNAseq reads (additional RNAseq sample preparation and analysis described below). We were able to detect sequencing reads mapping to CoV-NL63 or RSV genomes from the saliva of individual SS22 and SS23, respectively, so they were presumably infected with these pathogens at the time of saliva donation.

Saliva samples for apparently healthy individuals over a daily time course (SS26-SS32) were collected under a COVID-19-related sub-study of protocol 19-0696 (U. Colorado Boulder, PI Sawyer), where adult participants consented verbally and donated up to 2 mL of whole saliva per day of participation up to a total of 28 mL of whole saliva. The saliva was collected into Oragene saliva collection kit as mentioned above.

To purify RNA from saliva samples collected in Oragene saliva collection kit, we used 1 mL saliva 1:1 diluted in stabilization solution and followed the manufacturer recommended protocol by DNA Genotek to precipitate the nucleic acid. The RNA is further DNase-digested using Turbo DNase (Invitrogen #AM2238) and cleaned up using RNA clean-up and concentration micro-elute kit (Norgen #61000). The purified RNA is used for RT-qPCR or processed further for RNA-seq.

To prepare the total RNA for sequencing, we first spiked in ERCC RNA spike-in mix (ThermoFisher #4456740) into the saliva total RNA for downstream normalization. We depleted bacterial ribosomal RNA using pan-bacterial riboPOOL kit (siTOOLS #026). We then prepared the RNA for total RNA sequencing using KAPA RNA HyperPrep kit with RiboErase to remove human rRNA (Roche #KK8560). Finally, the saliva total RNA libraries were sequenced in 150 bp pair-end format using NovaSeq 6000 (Illumina) at the depth of 30 million reads.

Saliva samples for SARS-CoV-2-infected individuals (SS33-SS80), and matched SARS-CoV-2-negative individuals (SS81-SS100) were collected under protocol 20-0417 (U. Colorado Boulder, PI Sawyer), where adult participants 17 years of age or older (under a Waiver of Parental Consent) provided written consent. These samples were collected and tested for the SARS-CoV-2 virus during our campus COVID-19 testing initiative^{24,27} during the Fall 2020, Spring 2021, and Summer 2021 semesters. As part of this campus testing operation, university affiliates were asked to fill out a questionnaire to confirm that they did not present any symptoms consistent with COVID-19 at the time of sample donation, and to collect no less than 0.5 mL of saliva into a 5-mL screw-top collection tube. Saliva samples were heated at 95° C. for 30 min on site to inactivate the viral particles for safer handling, and then placed on ice or at 4° C. before being transported to the testing laboratory for RT-qPCR-based SARS-CoV-2 testing performed on the same day. Samples were then kept in −80 C until RNA preparation. The total RNA of the remaining saliva samples was then purified using TRIzol LS reagent (ThermoFisher #10296028) followed by GeneJET RNA cleanup and concentration kit (ThermoFisher #K0841). The purified total RNA was used for RT-qPCR following the steps described below.

Additional saliva samples for general assay development were collected under protocol 20-0068 (U. Colorado Boulder, PI Sawyer), where anonymous adult participants were verbally consented and donated up to 2 mL of whole saliva for use as a reagent in optimization and limit of detection experiments.

Analysis of High-Throughput Transcriptomics Data from Human Saliva Samples”

To profile human transcriptomic changes in human saliva samples, raw RNA sequencing reads in FASTQ format were obtained, trimmed using BBDuk (BBTools v38.05)⁴⁹, and mapped using HISAT2 v2.1.0⁵⁰ to human genome assembly hg38 along with ERCC spike-in sequence reference. Using NCBI RefSeq genome annotation (GRCh38.p13), we then counted the mapped reads assigned to gene or transcripts using FeatureCount (Subread v1.6.2)⁵¹. Read counts was first normalized using R package RUVseq (v1.28.0)⁵⁴ to account for library size factors based on the ERCC spike-in counts. Individual samples were then separated into infected and non-infected groups and the differential expression of genes were determined via DESeq2 (v3.1.3) Wald test⁵², from which the fold change and Benjamini-Hochberg adjusted p-values were obtained.

RT-qPCR Analysis of Universal Response Genes in Human Saliva:

Multiplex RT-qPCR analysis for the quantitative detection of human gene transcripts was carried out using customized and multiplexed TaqMan primer and probe mixes. Understanding that the contamination of genomic DNA often introduces quantification bias when measuring host gene expression, we explicitly designed primers that span exon junctions and limit the assay elongation time so that only the host RNA is reverse transcribed and amplified. As each transcript varies in its expression magnitude, we assigned genes into multiplex groups based on similar expression magnitudes observed in the meta-analysis of in vivo datasets and in human saliva. This minimizes competition of amplification reagents. Specifically, to determine the host gene expression levels, 1.5 μL of customized TaqMan multiplex probes were mixed with 5 μL 4×TaqPath 1-step multiplex master mix (ThermoFisher #A28526), 5 μL of saliva total RNA, and 8.5 μL of nuclease free water. The RT-qPCR assay was carried out on QuantStudio3 Real-time PCR system (ThermoFisher) consisting of a reverse transcription stage (25° C. for 2 min, 50° C. for 15 min, 95° C. for 2 min) followed by 45 cycles of PCR stage (95° C. for 3 s, 55° C. for 30 s, with a 1.6° C./s ramp-up and ramp-down rate). The cycle threshold (Ct) values were used to calculate relative fold change using delta delta Ct method. For the choice of internal control genes, we combined the meta-analysis (FIG. 29; cell culture experiments) and the saliva RNA-seq datasets (FIG. 30; human samples) to select genes for which the expression level remained most constant and abundant across the various conditions inherent to these experiments.

Infection of A549 Cells with Influenza a Virus:

For influenza A virus infection, human lung epithelial cells (A549s) where plated at a concentration of 1×10⁶ cells/well in a 6-well plate. The next day, the cells were infected with influenza A virus (Influenza A/Udorn/307/72) at an MOI=0.1 in serum-free media containing 1.0% bovine serum albumin. After 1 hour incubation, the inoculum was removed and replaced with growth media containing 1 ug/mL of N-acetylated trypsin. 24 hours post-infection, total RNA was harvest using QIAGEN RNeasy Mini kit (QIAGEN #74104).

Infection of Huh7 Cells with SARS-CoV-2:

Human Hepatoma (Huh7) cells (gift from Charles Rice, Rockefeller University) were grown in 1×DMEM (ThermoFisher cat. no. 12500062) supplemented with 2 mM L-glutamine (Hyclone cat. no. H30034.01), non-essential amino acids (Hyclone cat. no. SH30238.01), and 10% heat inactivated Fetal Bovine Serum (FBS) (Atlas Biologicals cat. no. EF-0500-A). The virus strain used for the assay was SARS-CoV2, USA WA January/2020, passage 3. Virus stocks were obtained from BEI Resources and amplified in Vero E6 cells to Passage 3 (P3) with a titer of 5.5×10⁵ PFU/mL. Cells were resuspended to 6.0×10⁵ cells/mL in 10% DMEM and seeded at 2 mL/well in 6-well plates. The plates were then incubated for approximately 24 hours (h) at 37° C., 5% CO₂ for cells to adhere prior to infection. Cell were infected with SARS-CoV-2 at an MOI of 0.01. Samples were harvested at 0, 2, 4, 8, 12, 24, and 48 hours post infection in 200 μl TRIzol reagent for RNA extractions following the manufacture's protocol.

The terminology used herein is for describing embodiments and is not intended to be limiting. As used herein, the singular forms “a,” “and” and “the” include plural referents, unless the content and context clearly dictate otherwise. Thus, for example, a reference to “a biomarker” may include a combination of two or more such biomarkers. Unless defined otherwise, all scientific and technical terms are to be understood as having the same meaning as commonly used in the art to which they pertain. As used herein, “about” or “approximately” means within 10% of a stated concentration range or within 10% of a stated time frame.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Nucleic acids and/or other moieties of the invention may be isolated or “extracted.” As used herein, “isolated” means separate from at least some of the components with which it is usually associated whether it is derived from a naturally occurring source or made synthetically, in whole or in part. Nucleic acids and/or other moieties of the invention may be purified. As used herein, purified means separate from the majority of other compounds or entities. A compound or moiety may be partially purified or substantially purified. Purity may be denoted by weight measure and may be determined using a variety of analytical techniques such as but not limited to mass spectrometry, HPLC, etc.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid but must be sufficiently complementary to hybridize with the template. The design of suitable primers for the amplification of a given target sequence is well known in the art and described in the literature cited herein.

As used herein, a biological marker (“biomarker” or “marker”) is a characteristic that is objectively measured and evaluated as an indicator of normal biologic processes, pathogenic processes, or pharmacological responses to therapeutic interventions, consistent with NIH Biomarker Definitions Working Group (1998). Markers can also include patterns or ensembles of characteristics indicative of particular biological processes. The biomarker measurement can increase or decrease to indicate a particular biological event or process. In addition, if the biomarker measurement typically changes in the absence of a particular biological process, a constant measurement can indicate occurrence of that process. In a preferred embodiment a biomarker includes one or more RNA transcripts that may be indicative of infection or other normal or abnormal physiological process.

As referred to herein, the terms “nucleic acid”, “nucleic acid molecules” “oligonucleotide”, “polynucleotide”, and “nucleotides” may interchangeably be used. The terms are directed to polymers of deoxyribonucleotides (DNA), ribonucleotides (RNA), and modified forms thereof in the form of a separate fragment or as a component of a larger construct, linear or branched, single stranded, double stranded, triple stranded, or hybrids thereof. The term also encompasses RNA/DNA hybrids. The polynucleotides may include sense and antisense oligonucleotide or polynucleotide sequences of DNA or RNA. The DNA molecules may be, for example, but not limited to: complementary DNA (cDNA), genomic DNA, synthesized DNA, recombinant DNA, or a hybrid thereof. The RNA molecules may be, for example, but not limited to: ssRNA or dsRNA and the like. The terms further include oligonucleotides composed of naturally occurring bases, sugars, and covalent internucleoside linkages, as well as oligonucleotides having non-naturally occurring portions, which function similarly to respective naturally occurring portions. The terms “nucleic acid segment” and “nucleotide sequence segment,” or more generally “segment,” will be understood by those in the art as a functional term that includes both genomic sequences, ribosomal RNA sequences, transfer RNA sequences, messenger RNA sequences, operon sequences, and smaller engineered nucleotide sequences that are encoded or may be adapted to encode, peptides, polypeptides, or proteins. All nucleic acid primers, such as SEQ IN NOs. 445-468, are presented in the 5′ to 3′ prime direction unless otherwise noted.

As used herein, “complementary” refers to the ability of a single strand of a polynucleotide (or portion thereof) to hybridize to an anti-parallel polynucleotide strand (or portion thereof) by contiguous base-pairing between the nucleotides (that is not interrupted by any unpaired nucleotides) of the anti-parallel polynucleotide single strands, thereby forming a double-stranded polynucleotide between the complementary strands. A first polynucleotide is said to be “completely complementary” to a second polynucleotide strand if each and every nucleotide of the first polynucleotide forms base-paring with nucleotides within the complementary region of the second polynucleotide. A first polynucleotide is not completely complementary (i.e., partially complementary) to the second polynucleotide if one nucleotide in the first polynucleotide does not base pair with the corresponding nucleotide in the second polynucleotide. The degree of complementarity between polynucleotide strands has significant effects on the efficiency and strength of annealing or hybridization between polynucleotide strands. This is of particular importance in amplification reactions, which depend upon binding between polynucleotide strands. An oligonucleotide primer is “complementary” to a target polynucleotide if at least 50% (preferably, 60%, more preferably 70%, 80%, still more preferably 90% or more) nucleotides of the primer form base-pairs with nucleotides on the target polynucleotide.

As referred to herein, the term “database” is directed to an organized collection of nucleotide sequence information that may be stored in a digital form. In some embodiments, the database may include any sequence information. In some embodiments, the database may include the genome sequence of a subject or a microorganism. In some embodiments, the database may include expressed sequence information, such as, for example, an EST (expressed sequence tag) or cDNA (complementary DNA) databases. In some embodiments, the database may include non-coding sequences (that is, untranslated sequences), such as, for example, the collection of RNA families (Rfam) which contains information about non-coding RNA genes, structured cis-regulatory elements and self-splicing RNAs. In exemplary embodiments, the databases may be selected from redundant or non-redundant GenBank databases (which are the NIH genetic sequence database, an annotated collection of all publicly available DNA sequences). Exemplary databases may be selected from, but not limited to: GenBank CDS (Coding sequences database), PDB (protein database), SwissProt database, PIR (Protein Information Resource) database, PRF (protein sequence) database, EMBL Nucleotide Sequence database, and the like, or any combination thereof.

As used herein, the term “detection” refers to the qualitative determination of the presence or absence of a microorganism in a sample. The term “detection” also includes the “identification” of a microorganism, i.e., determining the genus, species, or strain of a microorganism according to recognized taxonomy in the art and as described in the present specification. The term “detection” further includes the quantitation of a microorganism in a sample, e.g., the copy number of the microorganism in a microliter (or a milliliter or a liter) or a microgram (or a milligram or a gram or a kilogram) of a sample. The term “detection” also includes the identification of an infection in a subject or sample.

As used herein the term “pathogen” refers to an organism, including a microorganism, which causes disease in another organism (e.g., animals and plants) by directly infecting the other organism, or by producing agents that causes disease in another organism (e.g., bacteria that produce pathogenic toxins and the like). As used herein, pathogens include, but are not limited to bacteria, protozoa, fungi, nematodes, viroids and viruses, or any combination thereof, wherein each pathogen is capable, either by itself or in concert with another pathogen, of eliciting disease in vertebrates including but not limited to mammals, and including but not limited to humans. As used herein, the term “pathogen” also encompasses microorganisms which may not ordinarily be pathogenic in a non-immunocompromised host.

The term “infection,” or “infect” as used herein is directed to the presence of a microorganism within a subject body and/or a subject cell. For example, a virus may be infecting a subject cell. A parasite (such as, for example, a nematode) may be infecting a subject cell/body. In some embodiments, the microorganism may comprise a virus, a bacteria, a fungi, a parasite, or combinations thereof. According to some embodiments the microorganism is a virus, such as, for example, dsDNA viruses (such as, for example, Adenoviruses, Herpesviruses, Poxviruses), ssDNA viruses (such as, for example, Parvoviruses), dsRNA viruses (such as, for example, Reoviruses), (+) ssRNA viruses (+) sense RNA (such as, for example, Picornaviruses, Togaviruses), (−) ssRNA viruses (−) sense RNA (such as, for example, Orthomyxoviruses, Rhabdoviruses), ssRNA-RT viruses (+) sense RNA with DNA intermediate in life-cycle (such as, for example, Retroviruses), dsDNA-RT viruses (such as, for example, Hepadnaviruses). In some embodiments, the microorganism is a bacteria, such as, for example, a gram negative bacteria, a gram positive bacteria, and the like. In some embodiments, the microorganism is a fungi, such as yeast, mold, and the like. In some embodiments, the microorganism is a parasite, such as, for example, protozoa and helminths or the like. In some embodiments, the infection by the microorganism may inflict a disease and/or a clinically detectable symptom to the subject. In some embodiments, infection by the microorganism may not cause a clinically detectable symptom. In some embodiments, the microorganism is a symbiotic microorganism. In additional embodiments, the microorganism may comprise archaea, protists; microscopic plants (green algae), plankton, and the planarian. In some embodiments, the microorganism is unicellular (single-celled). In some embodiments, the microorganism is multicellular.

As used herein, the term “asymptomatic” refers to an individual who does not exhibit physical symptoms characteristic of being infected with a given pathogen, or a given combinations of pathogens.

The target biomarkers of this invention may be used for diagnostic and prognostic purposes, as well as for therapeutic, drug screening and patient stratification purposes (e.g., to group patients into a number of “subsets” for evaluation), as well as other purposes described herein.

Some embodiments of the invention comprise detecting in a sample from a patient, a level of a biomarker, wherein the presence or expression levels of the biomarker are indicative of infection or possible infection by one or more pathogens. As used herein, the term “biological sample” or “sample” includes a sample from any bodily fluid or tissue. Biological samples or samples appropriate for use according to the methods provided herein include, without limitation, blood, serum, urine, saliva, tissues, cells, and organs, or portions thereof. A “subject” is any organism of interest, generally a mammalian subject, and preferably a human subject.

Any isothermal amplification protocol can be used according to the methods provided herein. Exemplary types of isothermal amplification include, without limitation, nucleic acid sequence-based amplification (NASBA), loop-mediated isothermal amplification (LAMP), strand displacement amplification (SDA), helicase-dependent amplification (HDA), nicking enzyme amplification reaction (NEAR), signal mediated amplification of RNA technology (SMART), rolling circle amplification (RCA), isothermal multiple displacement amplification (EVIDA), single primer isothermal amplification (SPIA), recombinase polymerase amplification (RPA), and polymerase spiral reaction (PSR, available at nature.com/articles/srep12723 on the World Wide Web). In some cases, a forward primer is used to introduce a T7 promoter site into the resulting DNA template to enable transcription of amplified RNA products via T7 RNA polymerase. In other cases, a reverse primer is used to add a trigger sequence of a toehold sequence domain.

As used herein, the term “amplified” refers to polynucleotides that are copies of a particular polynucleotide, produced in an amplification reaction. An amplified product, according to the invention, may be DNA or RNA, and it may be double-stranded or single-stranded. An amplified product is also referred to herein as an “amplicon”. As used herein, the term “amplicon” refers to an amplification product from a nucleic acid amplification reaction. The term generally refers to an anticipated, specific amplification product of known size, generated using a given set of amplification primers.

TABLE 1
Comparison of gold standard tests to invention's lateral flow assay stick

| | | | Avg. | | | | |
|---|---|---|---|---|---|---|---|
| | | | time to | Able to | | | |
| | | | detection | detect | | | |
| Diagnostic | | | post- | unknown | | Trained | |
| Test Type | Sensitivity | Specificity | exposure | pathogens? | Laboratory? | personnel? | Cost |
| Serology- | High | Moderate | Late | No | Yes-most | Yes-most | $$ |
| based | | | | | cases | cases | |
| Cultures | Moderate | Moderate | Late | Only if | Yes | Yes | $$ |
| | | | | clinically | | | |
| | | | | suspected | | | |
| | | | | & able to be | | | |
| | | | | cultured | | | |
| PCR | High | High | Mid | No | Yes | Yes | $$$ |
| Our | High* | Moderate* | Earliest | Yes | No | No | $ |
| Product | | | | | | | |

TABLE 2
Primers used for the detection of SARS-CoV-2 (COVID-19)

| | | | | | SEQ ID |
|---|---|---|---|---|---|
| Name | Description | Oligonucleotide sequence (5′−>3′) | Label | Conc. | NO. |
| 2019-nCoV_N1-F | 2019-nCoV_N1 | 5′-GAC CCC AAA ATC AGC GAA | None | 20 μM | 469 |
| | Forward Primer | AT-3′ | | | |
| 2019-nCoV_N1-R | 2019-nCoV_N1 | 5′-TCT GGT TAC TGC CAG TTG | None | 20 μM | 470 |
| | Reverse Primer | AAT CTG-3′ | | | |
| 2019-nCoV_N1-P | 2019-nCoV_N1 | 5′-FAM-ACC CCG CAT TAC GTT | FAM, | 5 μM | 471 |
| | Probe | TGG ACC-BHQ1-3′ | BHQ-1 | | |
| 2019-nCoV_N2-F | 2019-nCoV_N2 | 5′-TTA CAA ACA TTG GCC GCA | None | 20 μM | 472 |
| | Forward Primer | AA-3′ | | | |
| 2019-nCoV_N2-R | 2019-nCoV_N2 | 5′-GCG CGA CAT TCC GAA | None | 20 μM | 473 |
| | Reverse Primer | GAA-3′ | | | |
| 2019-nCoV_N2-P | 2019-nCoV_N2 | 5′-FAM-ACA ATT TGC CCC CAG | FAM, | 5 μM | 474 |
| | Probe | CGC TTC AG-BHQ1-3′ | BHQ-1 | | |
| 2019-nCoV_N3-F | 2019-nCoV_N3 | 5′-GGG AGC CTT GAA TAC ACC | None | 20 μM | 475 |
| | Forward Primer | AAA A-3′ | | | |
| 2019-nCoV_N3-R | 2019-nCoV_N3 | 5′-TGT AGC ACG ATT GCA TTG- | None | 20 μM | 476 |
| | Reverse Primer | 3′ | | | |
| 2019-nCoV_N3-P | 2019-nCoV_N3 | 5′-FAM-AYC ACA TTG GCA CCC | FAM, | 5 μM | 477 |
| | Probe | GCA ATC CTG-BHQ1-3′ | BHQ-1 | | |
| RP-F | RNAse P Forward | 5′-AGA TTT GGA CCT GCG AGC | None | 20 μM | 478 |
| | Primer | G-3′ | | | |
| RP-R | RNAse P Reverse | 5′-GAG CGG CTG TCT CCA CAA | None | 20 μM | 479 |
| | Primer | GT-3′ | | | |
| RP-P | RNAse P | 5′-FAM - TTC TGA CCT GAA | FAM, | 5 μM | 480 |
| | Probe | GGC TCT GCG CG - BHQ-1-3′ | BHQ-1 | | |

TABLE 3
Transcriptomics datasets used for the discovery of human universal response genes

| | | | Virus, | Hours | |
|---|---|---|---|---|---|
| | | | Bacteria, | Post- | Sequencing |
| SRP Index | Human cell line | Pathogen | Fungus | Infection | Data Type |
| SRP044763 | IMR90 | Adenovirus | Virus | 24 | mRNA |
| SRP163661 | MRC5 | Adenovirus | Virus | 24 | Total |
| SRP202003 | HepG2 | Crimean-Congo hemorrhagic fever virus | Virus | 72 | Total |
| SRP078309 | A549 | Dengue Virus 2 | Virus | 36 | Total |
| SRP130978 | HUH751 | Dengue Virus 2 | Virus | NA | Total |
| SRP132737 | Huh7 | Dengue Virus 2 | Virus | 18 | Total |
| SRP188490 | HEK293 | Dengue Virus 2 | Virus | 18 | Total |
| SRP060253 | AGS | Ebola Virus | Virus | NA | Total |
| SRP101856 | DC | Ebola Virus | Virus | 24 | Total |
| SRP111145 | ARPE19 | Ebola Virus | Virus | 24 | Total |
| SRP255890 | B Cell | Ebola Virus | Virus | NA | Total |
| SRP272684 | B Cell Lymphoma | Ebola Virus | Virus | 24 | Total |
| SRP131318 | Rhabdomyosarcoma | Enterovirus | Virus | 6 | Total |
| SRP212863 | HUVEC | Hantaan Orthohantavirus | Virus | 72 | Total |
| SRP158789 | HepG2 | Hepatitis B Virus | Virus | 72 | Total |
| SRP187206 | HUH751 | Hepatitis C Virus | Virus | 148 | Total |
| SRP091538 | HepG2 | Hepatitis E Virus | Virus | 120 | Total |
| SRP117344 | KMB17 | Herpes Simplex Virus 1 | Virus | 48 | Total |
| SRP154536 | HEK293 | Herpes Simplex Virus 1 | Virus | 4 | Total |
| SRP163661 | MRC5 | Herpes Simplex Virus 1 | Virus | 9 | Total |
| SRP177947 | THP1 | Herpes Simplex Virus 1 | Virus | 24 | Total |
| SRP189489 | HFF | Herpes Simplex Virus 1 | Virus | 8 | Total |
| SRP065236 | HFF | Herpes Simplex Virus 2 | Virus | 8 | Total |
| SRP065236 | EC | Human Cytomegalovirus | Virus | 48 | Total |
| SRP065236 | HFF | Human Cytomegalovirus | Virus | 48 | Total |
| SRP065236 | NPC | Human Cytomegalovirus | Virus | 48 | Total |
| SRP163661 | MRC5 | Human Cytomegalovirus | Virus | 48 | Total |
| SRP266618 | NTT | Human Cytomegalovirus | Virus | 24 | Total |
| SRP065236 | CD4 + T Cell | Human Immunodeficiency Virus 1 | Virus | 120 | Total |
| SRP155217 | CD4 + T Cell | Human Immunodeficiency Virus 1 | Virus | 72 | Total |
| SRP155822 | Ileum organoid | Human Norovirus | Virus | 48 | Total |
| SRP223234 | HFK | Human Papillomavirus | Virus | NA | Total |
| SRP253951 | A549 | Human Parainfluenza Virus 3 | Virus | 24 | Total |
| SRP103819 | HNEpC | Human Rhinovirus | Virus | 48 | Total |
| SRP161185 | ATII | Influenza A Virus | Virus | 24 | Total |
| SRP230823 | HeLa | Influenza A Virus | Virus | 24 | Total |
| SRP234025 | A549 | Influenza A Virus | Virus | 48 | Total |
| SRP253951 | A549 | Influenza A Virus | Virus | 9 | Total |
| SRP272285 | A549 | Influenza A Virus | Virus | 6 | Total |
| SRP277269 | 293T | Influenza A Virus | Virus | 6 | Total |
| SRP281173 | A549 | Influenza A Virus | Virus | 12 | Total |
| SRP170549 | Calu3 | MERS-CoV | Virus | 24 | Total |
| SRP227272 | Calu3 | MERS-CoV | Virus | 24 | mRNA |
| SRP096169 | HFF | Orf Virus | Virus | 8 | Total |
| SRP277439 | HEK293 | Porcine Rubulavirus | Virus | 12 | Total |
| SRP229586 | A549 | Respiranny Syncytial Virus | Virus | 36 | Total |
| SRP229586 | H292 | Respiranny Syncytial Virus | Virus | 36 | Total |
| SRP229586 | HBEC | Respiranny Syncytial Virus | Virus | 36 | Total |
| SRP253951 | A549 | Respiranny Syncytial Virus | Virus | 24 | Total |
| SRP115192 | HSAEpC | Rift Valley Fever Virus | Virus | 18 | Total |
| SRP094462 | HInEpC | Rotavirus | Virus | 6 | Total |
| SRP253951 | A549-ACE2 | SARS-CoV-2 | Virus | 24 | Total |
| SRP270817 | PHAE | SARS-CoV-2 | Virus | 48 | Total |
| SRP273473 | DC | SARS-CoV-2 | Virus | 2 | Total |
| SRP273473 | MAC | SARS-CoV-2 | Virus | 2 | Total |
| SRP278618 | iPSC-derived | SARS-CoV-2 | Virus | 48 | Total |
| | cardiomyocyte | | | | |
| SRP081284 | MeWo | Varicella-zoster Virus | Virus | 24 | Total |
| SRP225661 | A549 | West Nile Virus | Virus | 24 | Total |
| SRP142592 | hNSC | Zika Virus | Virus | 72 | Total |
| SRP251704 | A549 | Zika Virus | Virus | 48 | Total |
| SRP253197 | HepG2 | Zika Virus | Virus | 48 | Total |
| SRP296743 | PBMC | Aspergillus fumigatus | Fungus | 24 | Total |
| SRP296743 | PBMC | Candida albicans | Fungus | 24 | Total |
| SRP296743 | PBMC | Rhizopus oryzae | Fungus | 24 | Total |
| SRP285913 | HeLa | Chlamydia trachomatis | Bacteria | 44 | Total |
| SRP321546 | DLD-1 | Fusobacterium nucleatum | Bacteria | 24 | Total |
| SRP321940 | Primly human | Listeria monocytogenes | Bacteria | 5 | Total |
| | trophoblasts | | | | |
| ERP020415 | THP-1 | Mycobacterium tuberculosis | Bacteria | 48 | Total |
| ERP115551 | hBMECs | Neisseria meningitidis | Bacteria | 6 | mRNA |
| SRP263458 | HUVEC | Staphylococcus aureus | Bacteria | 16 | Total |
| SRP072326 | A549 | Streptococcus pneumoniae | Bacteria | 2 | Total |

TABLE 4
The 69 universal response genes in humans.

| RefSeq Accession | Gene Symbol |
|---|---|
| NM_001547 | IFIT2 |
| NM_022168 | IFIH1 |
| NM_016323 | HERC5 |
| NM_014314 | DDX58 |
| NM_080657 | RSAD2 |
| NM_021127 | PMAIP1 |
| NM_001964 | EGR1 |
| NM_001945 | HBEGF |
| NM_005532 | IFI27 |
| NM_000584 | CXCL8 |
| NM_005252 | FOS |
| NM_014330 | PPP1R15A |
| NM_017414 | USP18 |
| NM_152542 | PPM1K |
| NM_014470 | RND1 |
| NM_006187 | OAS3 |
| NM_005101 | ISG15 |
| NM_001570 | IRAK2 |
| NM_001565 | CXCL10 |
| NM_022750 | PARP12 |
| NM_020529 | NFKBIA |
| NM_002463 | MX2 |
| NM_006820 | IFI44L |
| NM_001561 | TNFRSF9 |
| NM_006734 | HIVEP2 |
| NM_012420 | IFIT5 |
| NM_024119 | DHX58 |
| NM_021035 | ZNFX1 |
| NM_002228 | JUN |
| NM_017554 | PARP14 |
| NM_001432 | EREG |
| NM_012118 | NOCT |
| NM_003764 | STX11 |
| NM_002535 | OAS2 |
| NM_003733 | OASL |
| NM_003407 | ZFP36 |
| NM_007315 | STAT1 |
| NM_022147 | RTP4 |
| NM_004419 | DUSP5 |
| NM_017631 | DDX60 |
| NM_000958 | PTGER4 |
| NM_004420 | DUSP8 |
| NM_016584 | IL23A |
| NM_000201 | ICAM1 |
| NM_172140 | IFNL1 |
| NM_030641 | APOL6 |
| NM_002053 | GBP1 |
| NM_052941 | GBP4 |
| NM_002462 | MX1 |
| NM_138287 | DTX3L |
| NM_015907 | LAP3 |
| NM_005514 | HLA-B |
| NM_017633 | TENT5A |
| NM_003641 | IFITM1 |
| NM_001165 | BIRC3 |
| NM_002999 | SDC4 |
| NM_002038 | IFI6 |
| NM_004417 | DUSP1 |
| NM_001549 | IFIT3 |
| NM_006435 | IFITM2 |
| NM_006084 | IRF9 |
| NM_004335 | BST2 |
| NM_006509 | RELB |
| NM_080745 | TRIM69 |
| NM_033390 | ZC3H12C |
| NM_003141 | TRIM21 |
| NM_002176 | IFNB1 |
| NM_003745 | SOCS1 |
| NM_006417 | IFI44 |

TABLE 5
Human saliva samples used in this study

| Sample ID | Collection Date | Diagnosis / Infectious agent | Study Site |
|---|---|---|---|
| SS01-15 | March-December | Not detected, presumed healthy | University of Colorado |
| | 2019 | | Anschutz Medical School |
| | | | and Boulder |
| SS16 | September 2019 | Patient with gastroenteritis caused by Vibrio cholera. Received | University of Colorado |
| | | one dose of Cipro and ceftriaxone before saliva sample taken. | Anschutz Medical School |
| SS17 | September 2019 | Patient with Methicillin-resistant Staphylococcus aureus | |
| | | bacteremia and cervical osteomyelitis, discitis, and prevertebral | |
| | | abscess | |
| SS18 | September 2019 | Patient with VZV meningitis. Herpes Zoster involving left V1- | |
| | | V2 dermatome without ocular involvement | |
| SS19 | May, 2020 | Patient being treated for SARS-CoV-2 infection. Saliva | |
| SS20 | May, 2020 | samples taken several days (n = 4-7) following diagnosis. | |
| SS21 | May, 2020 | | |
| SS22 | Janualy, 2020 | University affiliates whose saliva contained RNAseq reads | University of Colorado |
| | | mapping to CoV-NL63 | Boulder |
| SS23 | Februaly, 2020 | University affiliates whose saliva contained RNAseq reads | |
| | | mapping to RSV | |
| SS24 | Feb, 2019 | Patient with Coccidioidomycosis (Valley Fever) | University of Colorado |
| SS25 | December, 2019 | Patient undergoing sepsis, likely 2.2 pyelonephritis by | Anschutz Medical School |
| | | Escherichia coli | |
| SS26-32 | May 2020- | 7 apparently healthy individuals who provided saliva samples | University of Colorado |
| | August 2020 | daily for 11 days | Boulder |
| SS33-80 | August 2020- | 48 covid-positive (but asymptomatic or pre-symptomatic) | |
| | December 2020 | university affiliates | |
| SS81-100 | | 20 covid-negative and apparently healthy university affiliates | |

TABLE 6
Top 30 differentially up- and down- regulated genes from
comparison between infected and healthy saliva

| Gene | Log2(Fold | Adjusted P- |
|---|---|---|
| Symbols | Change) | value |
| CHRNA5 | 6.05 | 9.35E−76 |
| IL2RA | 6.07 | 1.08E−71 |
| STS | 6.02 | 7.91E−69 |
| BAG5 | 5.80 | 9.31E−64 |
| HBD | 7.01 | 3.53E−53 |
| POR | 6.03 | 4.83E−50 |
| LCN10 | 6.38 | 4.06E−46 |
| C10orf55 | 7.06 | 9.76E−44 |
| TWIST1 | 6.35 | 1.08E−43 |
| CA2 | 6.97 | 1.19E−43 |
| NR0B1 | 7.13 | 7.96E−43 |
| GALE | 5.83 | 1.04E−42 |
| TENT5A | 6.15 | 2.69E−42 |
| WRN | 5.11 | 3.91E−42 |
| NOS3 | 5.95 | 5.09E−41 |
| HBEGF | 5.00 | 8.94E−41 |
| DRD4 | 6.13 | 5.62E−40 |
| NCMAP | 6.31 | 3.29E−39 |
| REN | 5.61 | 7.10E−39 |
| FGG | 4.98 | 2.07E−37 |
| HADHA | 5.01 | 8.57E−37 |
| HBG2 | 7.61 | 2.11E−36 |
| HOXD13 | 4.86 | 2.50E−36 |
| KITLG | 5.31 | 1.18E−35 |
| CHRNB1 | 5.74 | 1.08E−32 |
| ITGB3 | 4.59 | 2.63E−32 |
| BST2 | 6.03 | 3.66E−32 |
| OR56B1 | 7.34 | 4.66E−31 |
| HBG1 | 8.01 | 5.45E−31 |
| RND1 | 7.31 | 6.27E−31 |
| LOC102723665 | −3.38 | 1.86E−06 |
| GCSAM | −4.12 | 1.84E−05 |
| TAAR9 | −5.50 | 2.94E−05 |
| CDCA7L | −3.59 | 1.16E−04 |
| MIR320B2 | −4.81 | 1.47E−04 |
| HULC | −5.84 | 1.49E−04 |
| ZNF235 | −3.25 | 2.40E−04 |
| SLC39A12 | −3.05 | 3.28E−04 |
| IVNS1ABP | −3.87 | 3.58E−04 |
| KLHDC4 | −3.96 | 4.01E−04 |
| SERPINB5 | −3.57 | 4.41E−04 |
| L0C101927143 | −4.42 | 4.45E−04 |
| VAV2 | −3.29 | 4.68E−04 |
| DSEL | −4.39 | 5.69E−04 |
| RPL22 | −2.67 | 7.18E−04 |
| LINC01085 | −3.48 | 7.23E−04 |
| ERVW−1 | −3.94 | 8.02E−04 |
| SLC25A25−AS1 | −3.54 | 8.58E−04 |
| THOC5 | −2.59 | 9.56E−04 |
| UXT−AS1 | −4.49 | 1.21E−03 |
| TRI−AAT1−1 | −3.34 | 1.37E−03 |
| AKAP4 | −3.07 | 1.76E−03 |
| TADA2A | −2.58 | 2.03E−03 |
| LRRC7 | −3.49 | 2.71E−03 |
| LEMD1−AS1 | −3.55 | 3.02E−03 |
| GNG14 | −3.82 | 3.37E−03 |
| ZNF461 | −3.55 | 3.77E−03 |
| LINC01781 | −2.66 | 4.07E−03 |
| SAMD13 | −3.46 | 4.65E−03 |
| SLAMF8 | −1.81 | 5.00E−03 |

What is claimed is:

1. A method of detecting a host RNA transcript biomarker comprising the step of:
collecting a bodily fluid sample from a subject containing an RNA transcript biomarker;
converting said RNA transcript biomarker into a DNA probe, such as a double stranded DNA (dsDNA), single stranded DNA (ssDNA), or and a hybrid double stranded DNA (dsDNA) probe having:
a dsDNA target sequence;
a single stranded DNA (ssDNA) annealing region; and
a ssDNA target capture region;
introducing said hybrid dsDNA probe to a DNA conjugated reporter probe, wherein said ssDNA annealing region on hybrid dsDNA probe is complementary to a ssDNA annealing region of said DNA conjugated reporter probe such that the two probes are coupled together in a solution;
introducing the hybrid dsDNA probe and DNA conjugated reporter probe solution to a lateral flow assay test strip;
passing the solution through at least one detection zone on said lateral flow assay test strip, wherein said detection zone contains a plurality of embedded target capture probes having a ssDNA region that is complementary to said ssDNA target capture region on said hybrid dsDNA probe;
forming an immobilized complex aggregate comprising said hybrid dsDNA probe, said DNA conjugated reporter probe, and said target capture probe by annealing the complementary target capture region on said hybrid dsDNA probe with the target capture region on said target capture probe;
allowing a plurality of immobilized complex aggregates to form in said detection zone such that a detectable signal is produced.

2. The method of claim 1 wherein said bodily fluid sample comprises a saliva sample.

3. The method of claim 1 wherein said step of converting comprises the step of converting said RNA transcript biomarker into DNA probe through an isothermal reverse transcription recombinase polymerase amplification (RT-RPA) reaction.

4. The method of claim 3 wherein the reagents necessary to produce an isothermal reverse transcription recombinase polymerase amplification (RT-RPA) reaction are pre-loaded into a reaction cylinder.

5. The method of claim 1 wherein said dsDNA target sequence is coupled with said ssDNA annealing region and said ssDNA target capture region through a linker.

6. The method of claim 5 wherein said linker comprises a tri-carbon chain spacer (C3) linker.

7. The method of claim 1 wherein said DNA conjugated reporter probe comprises a conjugated gold nanoparticle (GNP) probe.

8. The method of claim 7 wherein said conjugated (GNP) probe comprises a GNP coupled to said ssDNA annealing region through a thiol, PEG₁₈, and PolyA construct.

9. The method of claim 1 wherein said target capture probe comprises a target capture probe having an immobilized streptavidin base tetramer coupled with a biotin-TEG linker that may further be coupled with said ssDNA target capture probe sequence that is complementary to said target capture region on said hybrid streptavidin.

10. The method of any of claims 1 and 8 wherein said lateral flow assay test strip further comprises:
a conjugate pad in fluid communication with a membrane that allows said solution to flow towards an absorbent pad via capillary action, wherein said absorbent pad is positioned distal to said detection zone.
a control zone that may immobilize unbound conjugated gold nanoparticle (GNP) probe

11. The method of claim 10 wherein said membrane comprises a nitrocellulose membrane.

12. The method of claim 1 wherein said RNA transcript biomarker comprises at least one RNA transcript biomarker encoded by at least one nucleotide sequence selected from the group consisting of: SEQ ID NO. 1-444, and 657-865.

13. A lateral flow assay for the early detection of RNA transcript biomarkers comprising:
a bodily fluid sample having a host RNA transcript biomarker from a subject;
a reaction cylinder configured to receive the saliva sample and further configured to generate an amplified sample through an isothermal reverse transcription recombinase polymerase amplification (RT-RPA) reaction wherein said amplified sample comprises a hybrid dsDNA probe coupled with a DNA conjugated reporter probe;
a conjugate pad configured to receive the amplified sample;
a membrane in fluid communication with said conjugate pad and further configured to allow said solution to flow through said membrane via capillary action;
a detection zone containing a plurality of embedded target capture probes configured to bind and immobilize said hybrid dsDNA probe;
a control zone configured to bind and immobilize one or more unbound DNA conjugated reporter probes; and
an absorbent pad positioned distal to said detection zone and said control zone.

14. The lateral flow assay of claim 13 wherein said bodily fluid sample comprises a saliva sample.

15. The lateral flow assay of claim 13 wherein the reagents necessary to produce said isothermal RT-RPA reaction are pre-loaded into said reaction cylinder.

16. The lateral flow assay of claim 13 wherein said membrane comprises a nitrocellulose membrane.

17. The lateral flow assay of claim 13 wherein said hybrid dsDNA probe comprises:
a dsDNA target sequence;
a ssDNA annealing region; and
a ssDNA target capture region.

18. The lateral flow assay of claim 17 wherein said ssDNA annealing region on hybrid dsDNA probe is complementary to a ssDNA annealing region of said DNA conjugated reporter probe, such that the two probes are coupled together in said amplified solution.

19. The lateral flow assay of claim 18 wherein said dsDNA target sequence is coupled with said ssDNA annealing region and said ssDNA target capture region through a linker.

20. The lateral flow assay of claim 19 wherein said linker comprises a tri-carbon chain spacer (C₃) linker.

21. The lateral flow assay of claim 13 wherein said DNA conjugated reporter probe comprises a conjugated gold nanoparticle (GNP) probe.

22. The lateral flow assay of claim 21 wherein said conjugated GNP probe comprises a GNP coupled to said ssDNA annealing region through a thiol, PEG₁₈, and PolyA construct.

23. The lateral flow assay of any of claims 13 and 17 wherein said target capture probes comprise a target capture probe having an immobilized streptavidin base tetramer coupled with a biotin-TEG linker that may further be coupled with said ssDNA target capture probe sequence that is complementary to said target capture region on said hybrid dsDNA probe.

24. The lateral flow assay of claim 13 wherein said host RNA transcript biomarker comprises at least one RNA transcript biomarker encoded by at least one nucleotide sequence selected from the group consisting of: SEQ ID NO. 1-444, and 657-865.

25. A antibody-based lateral flow assay for the early detection of RNA transcript biomarkers comprising:
a bodily fluid sample having a host RNA transcript biomarker from a subject;
a reaction cylinder configured to receive the saliva sample and further configured to generate an amplified sample through an isothermal reverse transcription recombinase polymerase amplification (RT-RPA) reaction wherein said amplified sample comprises a hybrid dsDNA probe coupled with an antibody conjugated reporter probe;
a conjugate pad configured to receive the amplified sample;
a membrane in fluid communication with said conjugate pad and further configured to allow said amplified sample to flow through said membrane via capillary action;
a detection zone containing a plurality of embedded antibody target capture probes configured to bind and immobilize said hybrid dsDNA probe;
a control zone containing a plurality of embedded antibody target capture probes configured to bind and immobilize said hybrid dsDNA probe;
a capture zone having an antibody configured to bind and immobilize one or more antibody DNA conjugated reporter probes.

26. The antibody-based lateral flow assay of claim 25 wherein said bodily fluid sample comprises a saliva sample.

27. The antibody-based lateral flow assay of claim 25 wherein the reagents necessary to produce said isothermal RT-RPA reaction are pre-loaded into said reaction cylinder.

28. The antibody-based lateral flow assay of claim 25 wherein said membrane comprises a nitrocellulose membrane.

29. The antibody-based lateral flow assay of claim 25 wherein said hybrid dsDNA probe comprises:
a dsDNA target sequence;
a 5′ forward ssDNA oligo; and
a 5′ reverse ssDNA oligo.

30. The antibody-based lateral flow assay of claim 29 wherein said 5′ forward ssDNA oligo comprises a 5′ FITC forward oligo.

31. The antibody-based lateral flow assay of claim 25 wherein said 5′ reverse ssDNA oligo comprises a 5′ DIG reverse oligo, or a 5′ Biotin reverse oligo.

32. The antibody-based lateral flow assay of claim 30 wherein said conjugated reporter probe comprises a gold nanoparticle (GNP) coupled with an antibody forming an antibody conjugated reporter probe.

33. The antibody-based lateral flow assay of claim 32 wherein said antibody comprises an anti-FITC antibody.

34. The antibody-based lateral flow assay of claims 30 and 33 wherein said FITC antibody binds to said 5′ FITC forward oligo of said hybrid dsDNA probe.

35. The antibody-based lateral flow assay of claim 25 wherein said target capture probe of said detection zone comprises an anti-DIG antibody.

36. The antibody-based lateral flow assay of claims 31 and 35 wherein said anti-DIG antibody binds to the 5′ DIG reverse oligo of said hybrid dsDNA probe.

37. The antibody-based lateral flow assay of claims 25 and 31 wherein said target capture probe of said control zone comprises a target capture probe having an immobilized streptavidin base tetramer coupled with a biotin-TEG linker that may further be coupled with said 5′ Biotin reverse oligo.

38. The antibody-based lateral flow assay of claim 30 wherein said target capture probe of said detection zone comprises an anti-rabbit antibody.

39. The antibody-based lateral flow assay of claim 25 wherein said host RNA transcript biomarker comprises at least one RNA transcript biomarker encoded by at least one nucleotide sequence selected from the group consisting of: SEQ ID NO. 1-444, and 657-865.

40. A method of early-pathogen detection comprising the step of:
collecting a bodily fluid sample from a first subject;
extracting host-derived biomarkers of infection and a pathogen biomarkers from said bodily fluid sample;
quantifying said host-derived biomarkers of infection and a pathogen biomarkers through PCR, real time PCR (RT-PCR), or quantitative real-time polymerase chain reaction (qRT-PCR);
establishing a time-course of the levels of host-derived biomarkers of infection and optionally correlating said host-derived biomarkers of infection with said levels of pathogen biomarkers in said bodily fluid sample;
optionally repeating the four above steps at different time-points;
collecting a bodily fluid sample from a second subject containing a host-derived biomarker of infection;
detecting one or more host-derived biomarkers of infection that correlate to infection with said pathogen.

41. The method of claim 40 wherein said bodily fluid sample comprises a saliva sample.

42. The method of claim 41 wherein said host-derived biomarkers of infection comprise host-derived RNA biomarkers of infection.

43. The method of claim 42 wherein said pathogen biomarkers comprises pathogen biomarkers selected from the group consisting of: viral pathogen biomarkers, bacterial pathogen biomarkers, and pathogen fungal biomarkers.

44. The method of claim 43 wherein said viral pathogen biomarkers comprise viral pathogen biomarkers from novel coronavirus SARS-CoV-2.

45. The method of claim 40 wherein said viral pathogen biomarkers from novel coronavirus SARS-CoV-2 comprises one or more biomarkers that may be amplified in a PCR reaction by the nucleotide primers according to SEQ ID NOs. 469-480.

46. The method of claim 40 wherein said host-derived biomarker of infection comprises host-derived RNA biomarkers of infection and further comprising the step of converting said host-derived RNA biomarkers of infection into a hybrid double stranded DNA (dsDNA) probe through an isothermal reverse transcription recombinase polymerase amplification (RT-RPA) reaction.

47. The method of claim 1 wherein said step of detecting comprises the method of claims 1-12.

48. A method of detecting an infection in a subject in need thereof, comprising the step of detecting at least one host-derived RNA biomarker of infection from a biological sample provided by said subject, wherein said at least one host-derived RNA biomarker of infection is selected from the group consisting of: a host-derived RNA biomarker of infection encoded by the nucleotide sequence according to SEQ ID NOs. 1-444, and 657-865.

49. The method of claim 48 wherein said step of detecting comprises the method of claims 1-12.

50. The method of claim 48 wherein said step of detecting comprises the step of detecting said host-derived RNA biomarker of infection comprises detecting a host-derived RNA biomarker of infection using PCR, RT-PCR, or qRT-PCR.

51. A lateral flow assay configured to detect at least one host-derived RNA biomarker from a biological sample provided by a subject, wherein said at least one host-derived RNA biomarker is selected from the group consisting of: a host-derived RNA biomarker encoded by the nucleotide sequence according to SEQ ID NOs. 1-444, and 657-865.

52. An assay configured to detect at least one host-derived RNA biomarker from a biological sample provided by a subject, wherein said at least one host-derived RNA biomarker is selected from the group consisting of: a host-derived RNA biomarker encoded by the nucleotide sequence according to SEQ ID NOs. 1-444, and 657-865, wherein said assay is a PCR assay, RT-PCR assay, or qRT-PCR assay.

53. A microarray assay configured to detect least one host-derived RNA biomarker from a biological sample provided by a subject, wherein said at least one host-derived RNA biomarker is selected from the group consisting of: a host-derived RNA biomarker encoded by the nucleotide sequence according to SEQ ID NOs. 1-444, and 657-865.

54. A lateral flow assay configured to detect at least one host-derived RNA biomarker indicative for a viral infection from a biological sample provided by a subject, wherein said at least one host-derived RNA biomarker indicative for a viral infection is selected from the group consisting of: IFIT2, ICAM1, ERG1, IFIH1, ISG15, CFB, CXCL10, DDX58, and IRAK2.

55. An assay configured to detect at least one host-derived RNA biomarker indicative for a viral infection from a biological sample provided by a subject, wherein said at least one host-derived RNA biomarker indicative for a viral infection is selected from the group consisting of IFIT2, ICAM1, ERG1, IFIH1, ISG15, CFB, CXCL10, DDX58, and IRAK2, wherein said assay is a PCR assay, RT-PCR assay, or qRT-PCR assay.

55. A microarray assay configured to detect least one host-derived RNA biomarker indicative for a viral infection from a biological sample provided by a subject, wherein said at least one host-derived RNA biomarker indicative for a viral infection is selected from the group consisting of: IFIT2, ICAM1, ERG1, IFIH1, ISG15, CFB, CXCL10, DDX58, and IRAK2.

56. A method of detecting a viral infection in a subject in need thereof, comprising detecting least one host-derived RNA biomarker indicative in a biological sample provided by a subject, wherein said at least one host-derived RNA biomarker indicative for a viral infection is selected from the group consisting of IFIT2, ICAM1, ERG1, IFIH1, ISG15, CFB, CXCL10, DDX58, and IRAK2, and said biological sample is saliva.

57. A lateral flow assay configured to detect at least one host-derived RNA biomarker indicative for a SARS-CoV-2 infection from a biological sample provided by a subject, wherein said at least one host-derived RNA biomarker indicative for a viral infection is selected from the group consisting of: MX1, PARP12, IFITM2, CD68, and SERINB3.

58. An assay configured to detect at least one host-derived RNA biomarker indicative for a SARS-CoV-2 infection from a biological sample provided by a subject, wherein said at least one host-derived RNA biomarker indicative for a viral infection is selected from the group consisting of MX1, PARP12, IFITM2, CD68, and SERINB3, wherein said assay is a PCR assay, RT-PCR assay, or qRT-PCR assay.

59. A microarray assay configured to detect least one host-derived RNA biomarker indicative for a SARS-CoV-2 infection from a biological sample provided by a subject, wherein said at least one host-derived RNA biomarker indicative for a viral infection is selected from the group consisting of: MX1, PARP12, IFITM2, CD68, and SERINB3.

60. A method of detecting a SARS-CoV-2 infection in a subject in need thereof, comprising detecting least one host-derived RNA biomarker in a biological sample provided by a subject, wherein said at least one host-derived RNA biomarker is indicative for a SARS-CoV-2 infection is selected from the group consisting of MX1, PARP12, IFITM2, CD68, and SERINB3, and said biological sample is saliva.

61. A lateral flow assay configured to detect at least one host-derived RNA biomarker indicative for an influenza infection from a biological sample provided by a subject, wherein said at least one host-derived RNA biomarker indicative for a viral infection is selected from the group consisting of: PLRG1, MSC, NKG7, NME8, and MMP12.

62. An assay configured to detect at least one host-derived RNA biomarker indicative for an influenza infection from a biological sample provided by a subject, wherein said at least one host-derived RNA biomarker indicative for a viral infection is selected from the group consisting of PLRG1, MSC, NKG7, NME8, and MMP12, wherein said assay is a PCR assay, RT-PCR assay, or qRT-PCR assay.

63. A microarray assay configured to detect least one host-derived RNA biomarker indicative for an influenza infection from a biological sample provided by a subject, wherein said at least one host-derived RNA biomarker indicative for a viral infection is selected from the group consisting of: PLRG1, MSC, NKG7, NME8, and MMP12.

64. A method of detecting an influenza infection in a subject in need thereof, comprising detecting least one host-derived RNA biomarker in a biological sample provided by a subject, wherein said at least one host-derived RNA biomarker is indicative for an influenza infection is selected from the group consisting of PLRG1, MSC, NKG7, NME8, and MMP12, and said biological sample is saliva.

65. The method of any of claims 51-64, wherein said RNA biomarker is selected from the group consisting of: a host-derived RNA biomarker encoded by the nucleotide sequence according to SEQ ID NOs. 1-444, and 657-865.

66. A nucleotide sequence encoding a host-derived RNA biomarker used to detect an infection in a subjected in need thereof, wherein said RNA biomarker is selected from the group consisting of: a nucleotide sequence according to SEQ ID NOs. 1-444, and 657-865.

67. A method of detecting a host-derived RNA biomarker comprising:
collecting a bodily fluid sample potentially containing a host-derived RNA biomarker and optionally a biomarker of a viral, bacterial, or fungal infection;
identifying a transcript of said host-derived RNA biomarker in the sample, and optionally a biomarker of a viral, bacterial, or fungal infection using a method selected from the group consisting of: PCR, RT-PCR, qPCR, transcript sequencing, a lateral flow assay, hybridization assay, microarray, nucleic acid detection assay.

68. The method of claim 67, wherein said bodily fluid sample comprises a saliva sample.

69. The method of claim 68, wherein said host-derived biomarkers of infection comprise host-derived RNA biomarkers of infection.

70. The method of claim 69, wherein said host-derived RNA biomarkers of infection comprises pathogen biomarkers selected from the group consisting of: viral pathogen biomarkers, bacterial pathogen biomarkers, and pathogen fungal biomarkers.

71. The method of claim 70, wherein said viral pathogen biomarkers comprise viral pathogen biomarkers from novel coronavirus SARS-CoV-2.

72. The method of claim 71, wherein said viral pathogen biomarkers from novel coronavirus SARS-CoV-2 comprises one or more biomarkers that may be amplified in a PCR reaction by the nucleotide primers according to SEQ ID NOs. 469-480.

73. The method of claim 69, wherein said host-derived biomarker of infection comprises host-derived RNA biomarkers of infection and further comprising the step of converting said host-derived RNA biomarkers of infection into a hybrid double stranded DNA (dsDNA) probe through an isothermal reverse transcription recombinase polymerase amplification (RT-RPA) reaction.

74. The method of claim 69, wherein said host-derived biomarker of infection comprises a host-derived RNA biomarker of infection is selected from the group consisting of: a host-derived RNA biomarker of infection encoded by the nucleotide sequence according to SEQ ID NOs. 1-444, and 657-865.

75. The method of claim 69, wherein said biomarker of a viral, bacterial, or fungal infection comprises an RNA biomarker of a viral, bacterial, or fungal infection.
